(19) United States
(12) Patent Application Publication
(10) Pub. No.: US 2012/0252119 A1
(43) Pub. Date: Oct. 4, 2012

(54) METHODS FOR THE COLLECTION AND MATURATION OF OOCYTES

(76) Inventors: Robert Bruce Gilchrist, Croydon (AU); Jeremy Thompson, Grange (AU); Firas Albuz, Jeddah (SA)

(21) Appl. No.: 13/320,245

(22) PCT Filed: May 14, 2010
(86) PCT No.: PCT/AU2010/000569

§ 371 Date: Jun. 20, 2012

Related U.S. Application Data
(60) Provisional application No. 61/178,318, filed on May 14, 2009.

(51) Int. Cl. C12N 5/07; C12N 5/02

(52) U.S. Cl. 435/375; 435/383; 435/384; 435/387; 435/404

(57) ABSTRACT

The present invention relates to a method of producing an embryo from an oocyte by an assisted reproduction technology. The method includes (a) collecting an oocyte from an ovary of a subject in a collection medium comprising a first phosphodiesterase inhibitor and an agent that increases intracellular cAMP concentration in the oocyte, (b) culturing the oocyte in a maturation medium comprising a second phosphodiesterase inhibitor, and (c) producing an embryo from the oocyte by an assisted reproduction technology. The present invention also relates to methods of inducing oocyte maturation. For example a method of in vitro maturation of an oocyte is described which comprises steps (a) and (b) above. The present invention also relates to an oocyte maturation medium comprising a phosphodiesterase inhibitor and a ligand for inducing maturation of the oocyte. A combination product comprising an oocyte collection and maturation medium referred to above is also described.

This international patent application claims priority from U.S. provisional patent application 61/178,318 filed on 14 May 2009, the contents of which are herein incorporated by this reference.

### FIELD OF THE INVENTION

The present invention relates generally to methods for the collection and maturation of oocytes. In particular, the present invention relates to in vitro methods that utilise improved collection and maturation media, which promote maturation of oocytes prior to fertilisation.

### BACKGROUND OF THE INVENTION

In mammals, immature eggs (oocytes) grow and develop in follicles within the ovary. Immature oocytes are metabolically coupled to somatic granulosa cells, which surround the oocyte and nurture the development of the oocyte until ovulation. Essentially, maturation of the oocyte depends on its association with its companion somatic granulosa cells which not only support its growth and development, but also regulate the progression of meiosis.

The cytoplasmic and nuclear maturation of the oocyte during pre-ovulatory development are closely related but differentially distinguishable processes crucial for successful fertilisation, development of the embryo, and possibly also for the ability of the embryo to implant, ultimately affecting pregnancy outcome.

During cytoplasmic development, the diameter of the oocyte substantially increases from ˜15 to 100 μm, corresponding to a 300-fold increase in volume. At this stage the oocyte is both transcriptionally and translationally very active. For example, a mature mouse oocyte contains ˜200-fold more RNA and ˜50-60-fold more protein than an average somatic cell. The content of mRNA in the oocyte is also high, ˜15-20%, compared to that of ˜2-3% in a somatic cell.

Nuclear maturation of the oocyte occurs after the gonadotropin luteinising hormone surge, and involves the dissolution of the nuclear membrane, chromosome condensation followed by orientation in the equatorial plate, and organisation of the microtubules in a spindle.

A significant proportion of children in western countries are now born using assisted reproduction technologies, including the use of in vitro fertilization (IVF). IVF generally takes the form of stimulating the ovaries of women to produce multiple growing follicles, collecting the ova from these large, growing follicles that are preparing to ovulate, contacting collected ova with sperm in vitro and introducing the resultant embryo into the uterus. In vitro maturation (IVM) of oocytes is an adjunct therapy to IVF, which greatly reduces the requirement for gonadotropin administration during treatment. IVM involves the removal of ova from smaller follicles in patients who receive either low levels of gonadotropin or even no gonadotropin. The procedure used to obtain eggs requires a modified patient management system and ova pick-up procedure.

Large doses of gonadotropin used in standard IVF procedures can lead to a condition of ovarian hyperstimulation syndrome (OHSS), which occurs in approximately 5% of women undertaking IVF cycles. OHSS is usually mild and self-limiting. In some cases, urgent medical attention is required. When severe, the condition can be potentially life threatening requiring hospitalization, intravenous fluids, pain relief, and other medication. Pulmonary embolism from a clot in the leg or complications of severe dehydration may occur in rare cases.

Women with the condition of polycystic ovarian syndrome require IVM in preference to IVF to avoid ovarian hyperstimulation caused by the administration of gonadotropin, or any other ovarian follicle stimulating agent. IVM is also applied to women whose preference is to minimise follicle stimulation during infertility treatment. IVM is also more convenient to the patient as it requires less drug administration, which is usually performed by the patients themselves. IVM also has cost advantages, as the cost of drug use is minimised.

Nevertheless, the efficiency of IVM relative to IVF in establishing pregnancies and live births are reduced. Although there have been some improvements in recent times to patient management, there has been little advance in laboratory techniques.

In vitro production (IVP) of animal embryos has a variety of purposes, such as genetic improvement in livestock and domesticated breeds, genetic rescue in rarer breeds, as well as a platform technology for manipulations, such as production of sexed embryos from sexed sperm, or cloning by somatic cell nuclear transfer. An essential technique in the production of embryos in vitro is the maturation of oocytes in vitro (IVM). IVP has the potential to replace current conventional techniques such as multiple ovulation and embryo transfer (MOET), where (similarly to human clinical application) gonadotropin treatment is required. However, adoption of IVP for breeding and other uses has been hampered by the poor efficiencies of producing transferable stage embryos, the poor results following embryo transfer of such embryos and the poor results following freezing and thawing (storage) of such embryos.

Accordingly, new methods and media for culturing oocytes would be desirable. In particular, new methods and media for collecting and maturing oocytes to improve assisted reproductive technologies would be particularly desirable.

Reference to any prior art in this specification is not, and should not be taken as, an acknowledgment or any form of suggestion that this prior art forms part of the common general knowledge in any country.

### SUMMARY OF THE INVENTION

The present invention arises out of studies of oocyte in vitro culture media, and the constituents thereof, which enhance the maturation of oocytes once harvested from the ovary.

In one aspect, the present invention provides a method of producing an embryo from an oocyte by an assisted reproduction technology, the method comprising:
(a) collecting an oocyte from an ovary of a subject in a collection medium comprising a first phosphodiesterase inhibitor and an agent that increases intracellular cAMP concentration in the oocyte;
(b) culturing the oocyte in a maturation medium comprising a second phosphodiesterase inhibitor; and
(c) producing an embryo from the oocyte by an assisted reproduction technology.

In another aspect, the present invention provides a method of in vitro maturation of an oocyte, the method comprising:
(a) collecting an oocyte from an ovary of a subject in a collection medium comprising a first phosphodiesterase inhibitor and an agent that increases intracellular cAMP concentration in the oocyte; and
(b) culturing the oocyte in a maturation medium comprising a second phosphodiesterase inhibitor.

In another aspect, the present invention provides an oocyte maturation medium, the medium comprising:
(a) a phosphodiesterase inhibitor; and
(b) a ligand for inducing maturation of the oocyte,
wherein the concentration of the ligand in the oocyte maturation medium overcomes cAMP-induced meiotic arrest of the oocyte.

In another aspect, the present invention provides a combination product comprising the following components:
(a) an oocyte collection medium comprising a first phosphodiesterase inhibitor and an agent that increases intracellular cAMP concentration in the oocyte; and
(b) an oocyte maturation medium comprising a second phosphodiesterase inhibitor and a ligand for inducing maturation of the oocyte;
wherein the concentration of the ligand in the oocyte maturation medium overcomes cAMP-induced meiotic arrest of the oocyte.

In another aspect, the present invention provides a method of inducing oocyte maturation, the method comprising culturing an oocyte in a maturation medium comprising a phosphodiesterase inhibitor and a ligand for inducing maturation of the oocyte, wherein the concentration of the ligand in the maturation medium overcomes cAMP-induced meiotic arrest of the oocyte, thereby maturing the oocyte.

In another aspect, the present invention provides a method of inducing maturation of an oocyte which is in a state of meiotic arrest, the method comprising contacting the oocyte with a ligand at a concentration sufficient to overcome the meiotic arrest.

### BRIEF DESCRIPTION OF THE FIGURES

FIG. 1 are graphs showing the effect of increasing doses of forskolin without (A) or with (B) IBMX (500 μM) during pre-IVM phase on the cAMP content of the whole COCs (oocytes with their cumulus vestments intact) at the end of pre-IVM.

FIG. 2 is a graph demonstrating the effect of pre-IVM duration on COC cAMP when incubated with increasing concentrations of forskolin and IBMX.

FIG. 3 is a graph showing the effect of pre-IVM duration on intra-oocyte cAMP content—those collected and incubated with their cumulus vestments intact (COCs) and denuded prior to assay (DO).

FIG. 4 are graphs showing the effect of various pre-in vitro maturation (pre-IVM) phase treatments over time on the cAMP content of two different types of oocytes: (A) oocytes collected and assayed with their cumulus vestments intact (COC); and (B) oocytes collected as COCs but denuded of their cumulus vestment prior to assay (DO).

FIG. 5 is a graph showing the effect on spontaneous oocyte maturation (GV/GVBD) following incubation of cumulus-oocyte complexes in various pre-in vitro maturation (pre-IVM) and IVM phase media.

FIG. 6 is a series of charts showing the effect on germinal vesicle (GV) configurations of cumulus-oocyte complexes cultured in various pre-IVM and IVM phase media.

FIG. 7 is a graph showing the effect on oocyte-cumulus cell gap junctional communication following incubation of oocytes in various pre-IVM and IVM phase media.

FIG. 8 is charts showing the effect of follicle stimulating hormone (FSH) on inducing meiotic maturation of cumulus-oocyte complexes (COCs) exposed to various pre-IVM and IVM phase media.

FIG. 9 is a graph showing the intracellular cAMP concentration of oocytes exposed to various pre-IVM and IVM phase media.

FIG. 10 is a graph showing the effect of an epidermal growth factor receptor (EGFR) inhibitor on follicle stimulating hormone (FSH)-induced oocyte maturation in the presence of a type-3 PDE inhibitor.

FIG. 11 are graphs demonstrating the effect of IBMX and increasing doses of forskolin in the pre-IVM phase on oocyte developmental capacity when oocytes are matured during the IVM phase in the presence of 20 μM cilostamide.

FIG. 12 shows two graphs summarising the effect of the presence of cAMP modulating agents present in various pre-IVM and IVM phase media on oocyte developmental capacity (i.e. cleavage and development to the blastocyst stage).

FIG. 13 is a graph showing the effect of the presence of cAMP modulating agents present in various pre-IVM and IVM phase media on blastocyst cell numbers.

FIG. 14 are graphs showing a comparison between in vivo cAMP concentrations in mouse cumulus-oocyte complexes (COCs) following follicular growth induced by equine chorionic gonadotropin (eCG) and human chorionic gonadotropin (hCG)-induced oocyte maturation (A) compared with cAMP concentrations in in vitro cultured COCs during pre-IVM phase (2 hours which includes COC collection and selection) before oocyte in vitro maturation (IVM) (B).

FIG. 15 are graphs showing the effect of increasing doses of FSH to induce meiotic maturation of cumulus-oocyte complexes (COCs) matured in the presence of the type-3 PDE inhibitor (cilostamide; 1 μM (A) or 0.1 μM (B)).

FIG. 16 is a graph showing the effect of cAMP modulators during pre-IVM and IVM on the time it takes to complete oocyte meiotic maturation.

FIG. 17 is a graph showing the meiotic maturation of mouse COCs matured either in induced IVM or spontaneous IVM following 18 and 22 hours of culture.

FIG. 18 shows two graphs demonstrating the effect of maturing mouse oocytes in vitro (in either spontaneous or induced IVM either for 18 or 22 hours of IVM) on oocyte developmental capacity as measured by cleavage rate (day 2)(A) and blastocyst rate (day 5)(B).

FIG. 19 includes graphs that show the effect of spontaneous IVM compared to induced IVM on mouse blastocyst quality.

FIG. 20 is a graph that shows the effect of using different cAMP modulators during the pre-IVM and IVM phases on mouse oocyte developmental capacity.

FIG. 21 are graphs showing the developmental competence of mouse oocytes matured either in vivo, by induced IVM or by spontaneous IVM.

FIG. 22 provides graphs showing the effect of induced IVM on pregnancy outcomes (A-C) and on fetal development (D-E) in mice transferred with embryos derived from oocytes that were matured in vivo, by induced IVM or by spontaneous IVM.

FIG. 23 is an illustration summarising the key concepts of induced IVM compared to conventional IVF (in vivo matured oocytes) and standard spontaneous IVM and the relative efficiencies of these three procedures at generating fetuses.

### GENERAL DESCRIPTION OF THE INVENTION

Where the terms “comprise”, “comprises”, “comprised” or “comprising” are used in this specification (including the claims) they are to be interpreted as specifying the presence of the stated features, integers, steps or components, but not precluding the presence of one or more other feature, integer, step, component or group thereof.

As used in this specification, the singular forms “a”, “an” and “the” include plural aspects unless the context clearly dictates otherwise.

Where a range of values is expressed, it will be clearly understood that this range encompasses the upper and lower limits of the range, and all values in between these limits.

The present invention relates to an improved method of collection and maturation of oocytes in vitro. It has been determined that maturation of oocytes harvested from the ovary in a number of species, including human and bovine species, is significantly improved when the harvested oocyte is placed in a collection medium comprising a phosphodiesterase inhibitor and an agent that increases intracellular cAMP concentration in the oocyte and/or in cumulus cells associated with the oocyte, followed by culturing the oocyte in a maturation medium also comprising a phosphodiesterase inhibitor. With respect to assisted reproduction technologies, the improved methodology allows fertilisation of the oocyte to be delayed until the maturity of the oocyte is closer to that occurring naturally during the reproductive cycle (when compared to the maturity of oocytes collected in known media).

Accordingly, in a first aspect the present invention provides a method of producing an embryo from an oocyte by an assisted reproduction technology, the method comprising:
(a) collecting an oocyte from an ovary of a subject in a collection medium comprising a first phosphodiesterase inhibitor and an agent that increases intracellular cAMP concentration in the oocyte;
(b) culturing the oocyte in a maturation medium comprising a second phosphodiesterase inhibitor; and
(c) producing an embryo from the oocyte by an assisted reproduction technology.

Furthermore, in a second aspect the present provides a method of in vitro maturation of an oocyte, the method comprising:
(a) collecting an oocyte from an ovary of a subject in a collection medium comprising a first phosphodiesterase inhibitor and an agent that increases intracellular cAMP concentration in the oocyte; and
(b) culturing the oocyte in a maturation medium comprising a second phosphodiesterase inhibitor.

As discussed above, the success of the assisted reproductive technology depends to a large extent on the maturity of the oocyte prior to fertilisation. Oocytes harvested from ovaries typically undergo spontaneous resumption of meiosis, i.e. proceed to nuclear maturation, when placed in culture. This nuclear maturation may often occur before the oocyte has undergone complete cytoplasmic maturity. This is believed to ultimately affect the success of fertilisation and possibly subsequent embryo implantation and development.

In this regard, it will be understood that the term “oocyte” includes an oocyte alone or an oocyte in association with one or more other cells, such as an oocyte as part of a cumulus oocyte complex.

Accordingly, the methods according to the first and second aspects of the invention utilise a media for collecting an oocyte from an ovary of a subject, also referred to herein as “an oocyte collection medium”, “collection medium”, or variations thereof, which comprises a first phosphodiesterase inhibitor and an agent that increases intracellular cAMP concentration in the oocyte.

Furthermore, the methods according to the first and second aspects of the invention utilise a media for subsequent culturing and maturing of the collected oocyte, also referred to herein as “an oocyte maturation medium”, “maturation medium”, or variations thereof, which comprises a second phosphodiesterase inhibitor.

As set out above, both the collection and maturation media contain a phosphodiesterase inhibitor. The presence of a phosphodiesterase inhibitor in the collection and maturation media, and the further presence of an agent which increases oocyte intracellular cAMP in the collection medium, provides the advantage of preventing the harvested oocyte from undergoing spontaneous resumption of meiosis. Therefore the respective media promote cytoplasmic maturation of the oocyte before the nuclear maturation and subsequent fertilisation process begins.

A “phosphodiesterase inhibitor” is to be understood to mean an agent which blocks or inhibits phosphodiesterases (PDEs) directly or indirectly and whose action results in inactivation of the cyclic nucleotide targets (for example, cAMP and cGMP) by hydrolytic cleavage of the 3′-phosphodiester bond, resulting in passive accumulation of specific cyclic nucleotides. Inhibitors can be non-selective for all phosphodiesterase isoforms or selective for specific isoforms.

“Phosphodiesterase isoforms” refer to a family of isozymes or isoforms responsible for the metabolism or degradation of the intracellular second messengers, cAMP and cGMP. Specific isoforms can have highly selective cellular and subcellular localizations. Examples of phosphodiesterase isoforms include PDE3 and PDE4.

PDE inhibitors which can be used in the methods of the invention include any non-toxic inhibitor of PDE, whether selective or non-selective in nature. PDE inhibitors may be in the form of proteins, antibodies, aptamers, antisense nucleic acids, antisense oligonucleotides, siRNAs, polypeptides, peptides, small molecules, drugs, polysaccharides, glycoproteins, and lipids. For example, suitable PDE inhibitors include, but are not limited to, isobutylmethylxanthine (IBMX), cilostamide, theophylline, AH-21-132, Org-30029 (Organon), Org-20241 (Organon), Org-9731 (Organon), Zardaverine, vinpocetine, EHNA (MEP-1), Milrinone, Siguazodan, Zaprinast, SK+F 96231, Tolafentrine (Byk Gulden), and Filaminast (Wyeth-Ayerst Pharmaceuticals). Other PDE inhibitors are also known in the art.

The PDE inhibitor in the collection medium (“the first PDE inhibitor”) may be the same as, or different to, the PDE inhibitor in the maturation medium (“the second PDE inhibitor”).

In one embodiment, the PDE inhibitor in the collection medium is IBMX.

In another embodiment, the PDE inhibitor in the maturation medium is cilostamide.

In one specific embodiment, the PDE inhibitor in the collection medium is IBMX and the PDE inhibitor in the maturation medium is cilostamide.

As set out above, the collection media also contains an agent that increases the concentration or level of intracellular cAMP in the collected oocyte and/or increases the concentration or level of cAMP in cumulus cells associated with the oocyte. The agent may do so directly or indirectly by increasing cAMP synthesis or production, or by decreasing its degradation, or both, within the oocyte and/or in cumulus cells associated with the oocyte. Methods for measuring the level of cAMP synthesis, production or degradation are known in the art.

In one embodiment, cAMP synthesis or production may be increased by at least 10%, 20%, 30%, 40%, 50%, 60%, 70%, 80%, 90%, 100%, 2-fold, 5-fold, 10-fold, 20 fold, 50-fold, or 100-fold, relative to an untreated oocyte. Similarly, in another embodiment, cAMP degradation may be decreased by at least 10%, 20%, 30%, 40%, 50%, 60%, 70%, 80%, 90%, or 100, relative to an untreated oocyte.

Agents that increase the concentration or level of intracellular cAMP may be in the form of proteins, antibodies, aptamers, antisense nucleic acids, antisense oligonucleotides, siRNAs, polypeptides, peptides, small molecules, drugs, polysaccharides, glycoproteins, and lipids. For example, agents which increase the synthesis or production of cAMP include activators of adenyl cyclase, such as forskolin. Examples of modulators which decrease cAMP degradation include inhibitors of phosphodiesterases such as theophylline. In one embodiment, the agent which increases the concentration or level of intracellular cAMP is one or more of forskolin, invasive adenylate cyclase and prostaglandin E₂.

The method according to the first and second aspects of the invention may further include the step of exposing the oocyte to a ligand which induces nuclear maturation of the oocyte. In one embodiment, the concentration of the ligand is sufficient to overcome cAMP-induced meiotic arrest of the oocyte.

The ligand may be included as a component of the oocyte maturation medium, or may be a separate component or part of a separate media to which the oocyte is contacted with. In the latter case, the ligand may be added after step (b) of the methods according to the first and second aspects of the invention.

The ligand may be in the form of proteins, antibodies, aptamers, antisense nucleic acids, antisense oligonucleotides, siRNAs, polypeptides, peptides, small molecules, drugs, polysaccharides, glycoproteins, and lipids. For example, the ligand may include, but is not limited to, follicle stimulating hormone (FSH), epidermal growth factor (EGF) (including the EGF-like peptides amphiregulin and epiregulin), or functional isoforms thereof. It is contemplated that one or more ligands may be used in the methods of the invention.

In one embodiment, the ligand is FSH.

In one embodiment, the concentration of FSH is greater than 10 mlU/ml. For example, the concentration of FSH may be in the range between 10-200 mlU/ml.

In a further embodiment, the ligand is EGF.

In one embodiment, the concentration of EGF is greater than 1 ng/ml.

The first aspect of the invention contemplates an “assisted reproductive technology”. The term “assisted reproduction technology” as used throughout the specification is to be understood to mean any fertilization technique in humans and animals involving isolated oocytes and/or isolated sperm, including a technique using an oocyte or embryo cultured in vitro (for example in vitro maturation of an oocyte), in vitro fertilization (IVF; aspiration of an oocyte, fertilization in the laboratory and transfer of the embryo into a recipient), gamete intrafallopian transfer (GIFT; placement of oocytes and sperm into the fallopian tube), zygote intrafallopian transfer (ZIFT; placement of fertilized oocytes into the fallopian tube), tubal embryo transfer (TET; the placement of cleaving embryos into the fallopian tube), peritoneal oocyte and sperm transfer (POST; the placement of oocytes and sperm into the pelvic cavity), intracytoplasmic sperm injection (ICSI), testicular sperm extraction (TESE), and microsurgical epididymal sperm aspiration (MESA); or any other in vitro technique for producing embryos in humans and/or animals, such as nuclear transfer, parthenogenic activation and the use of totipotent cells.

In one embodiment, the assisted reproduction technology is used to produce a human embryo.

In another embodiment, the assisted reproduction technology is used to produce a bovine embryo.

In a method according to a first and second aspect of the invention an oocyte is first harvested or collected from an ovary of a subject. Oocyte collection can be performed according to standard techniques long known in the art. For example, see Textbook of Assisted Reproduction: Laboratory and Clinical Perspectives (2003) Editors Gardner, D. K., Weissman, A., Howles, C. M., Shoham, Z. Martin Dunits Ltd, London, UK; and Gordon, I. (2003) Laboratory Production of Cattle Embryos 2nd Edition CABI Publishing, Oxon, UK.

Most oocyte collection techniques involve the insertion of an aspirating needle into an ovarian follicle using transvaginal ultrasound. The aspirating needle is connected by tubing to a material collection trap and the collection trap, in turn, is connected to a suction source such as a manually operated syringe or an electromechanical vacuum source. Oocytes are typically isolated from multiple follicles. As such, the harvested oocytes represent a heterogenous population with regard to their developmental potential.

In one embodiment of the methods of the invention, the assisted reproduction technology comprises IVF. IVF relates to the fertilization of an oocyte in vitro, wherein the oocyte is isolated from the subject and incubated in liquid media to allow fertilization of the oocyte.

As indicated above, methods are well known in the art for collecting oocytes from suitable females and fertilizing the oocytes in vitro. It is contemplated that fertilisation of the oocyte will ideally occur greater than 24 hours, but no later than 60 hours, after the oocyte collection step, such that maturity of the oocyte is at a sufficient stage to maximise the success of subsequent steps in the IVF procedure.

Typically, the oocyte is held in the collection medium for 15-120 minutes at 35-39° C. The oocyte is then incubated in the maturation medium for 20-60 hours, typically in a 37-39° C. incubator with a suitable gas mixture. An example of a suitable gas mixture includes, but is not limited to, a gas mixture comprising of CO₂ (1-10% by volume), balanced with air or with mixtures of O₂ and N₂ in proportions that sustain biological activity.

The oocyte is then held in the maturation medium for between 16 and 60 hours of maturation, typically between 24-50 hours of maturation and in some cases between 28-44 hours of maturation.

As will be appreciated, the time for maturation may differ between species. Generally the time of maturation will be the time that the meiotic stage of metaphase II is reached in these systems, and as such the time for maturation will typically be from 12 hours before to 18 hours after the median time to metaphase II stage of meiosis is reached. A suitable time would be from 3 hours before and 6 hours after the meiotic stage of metaphase II is reached. For example in the bovine setting the IVM time will generally be in the range from 18-24 hours in the absence of compounds that specifically inhibit meiosis progression to metaphase II.

In the human setting, the IVM time will generally be greater than 30 hours, and most usually between 30-50 hours.

In one embodiment, the IVM time in human is equal to or greater than 36 hours, for example between 36-48 hours.

In one specific embodiment, the IVM time in the human is equal to or greater than 40 hours, for example between 40-48 hours.

In one specific embodiment, the IVM time in the human is equal to or greater than 48 hours, for example between 48-50 hours.

The term “subject” as used throughout the specification is to be understood to include any female subject including a female human or a female mammal. Examples of suitable mammals include a primate, a livestock animal (e.g., a horse, a cow, a sheep, a pig, or a goat), a companion animal (e.g. a dog or a cat), a laboratory test animal (e.g. a mouse, a rat, or a guinea pig), or any animal of veterinary or economic significance.

In one embodiment, the subject is a Bos indicus cow. In another embodiment, the subject is a Bos taurus cow.

In these and other aspects of the invention, the oocyte may be, for example, an oocyte that is part of a follicle, part of a cumulus oocyte complex (COO) or may be a denuded oocyte.

In one embodiment of the methods of the invention, the subject is a female human, the oocyte is a human oocyte, and the embryo is a human embryo.

In another embodiment of the methods of the invention, the subject is a cow, the oocyte is a bovine oocyte, and the embryo is a bovine embryo.

It will be appreciated that a phosphodiesterase inhibitor, an agent that increases intracellular cAMP concentration in the oocyte, and a ligand which induces nuclear maturation of the oocyte, may be used as supplements in oocyte culture media, including oocyte maturation media.

Accordingly, in a third aspect the present invention provides an oocyte culture medium comprising:
(a) a phosphodiesterase inhibitor; and
(b) a ligand for inducing nuclear maturation of the oocyte.

In one embodiment, the oocyte culture medium is an oocyte maturation medium.

In a fourth aspect the present invention provides an oocyte maturation medium comprising:
(a) a phosphodiesterase inhibitor; and
(b) a ligand for inducing nuclear maturation of the oocyte,
wherein the concentration of the ligand in the oocyte maturation medium overcomes cAMP-induced meiotic arrest of the oocyte.

In some embodiments, the phosphodiesterase inhibitor in the maturation medium according to the third or fourth aspect of the invention may be as hereinbefore described. In one specific embodiment, the phosphodiesterase inhibitor may be cilostamide.

In some embodiments, the ligand according to the third or fourth aspect of the invention may be as hereinbefore described. In specific embodiments, the ligand may be FSH or EGF. The concentration of each ligand may be as hereinbefore described.

In some embodiments of the third and fourth aspects of the invention, the oocyte maturation medium is a human oocyte maturation medium or a bovine oocyte maturation medium.

In a fifth aspect, the present invention provides a combination product comprising the following components:
(a) an oocyte collection medium comprising a first phosphodiesterase inhibitor and an agent that increases intracellular cAMP concentration in the oocyte; and
(b) an oocyte maturation medium comprising a second phosphodiesterase inhibitor and a ligand for inducing maturation of the oocyte;
wherein the concentration of the ligand in the oocyte maturation medium overcomes cAMP-induced meiotic arrest of the oocyte.

In one embodiment of this aspect of the invention, the agent increases intracellular cAMP production in the oocyte. For example, the agent may be forskolin. In another embodiment, the agent decreases intracellular cAMP degradation in the oocyte.

In some embodiments, the ligand in the maturation medium is FSH or EGF. The concentration of FSH is generally greater than 10 mlU/ml and the concentration of EGF is generally greater than 1 ng/ml.

In some embodiments of the fifth aspect of the invention, the first phosphodiesterase inhibitor and the second phosphodiesterase inhibitor are different. For example, the first phosphodiesterase inhibitor may be IBMX and the second phosphodiesterase inhibitor may be cilostamide.

In some embodiments, the first phosphodiesterase inhibitor and the second phosphodiesterase inhibitor are the same.

In some embodiments, the oocyte collection and maturation medium, or combination product thereof, of the invention may be used according to any one of the methods of the invention. For example, they may be used for collection and maturation of a human or a bovine oocyte.

The oocyte collection medium of the present invention may also be used for flushing, washing and holding the oocyte during the process of harvesting the oocyte from an ovary of the subject.

The oocyte collection medium may be comprised of one or more of NaCl, KCl, Mg₂SO₄, KH₂PO₄, Ca[lactate], NaHCO₃, amino acids and derivatives, a protein such as serum albumin, glucose, pyruvic acid and an antibiotic. The medium includes a PDE inhibitor and an agent which increases the intracellular concentration of cAMP in the oocyte, as hereinbefore described.

In some embodiments, the PDE inhibitor in the oocyte collection medium is IBMX. In one embodiment, the concentration of IBMX is in the range between 5-5000 μM. Generally the concentration is in the range between 50-1000 μM.

In some embodiments, the agent which increases the intracellular concentration of cAMP in the oocyte is forskolin. In one embodiment, the concentration of forskolin is in the range from 1-2000 μM. Generally the concentration is in the range between 10-200 μM.

The oocyte maturation medium of the present invention allows maturation of the collected oocyte to a physical stage prior to fertilisation which simulates the maturity of an oocyte which is released by the ovary during ovulation in the reproductive cycle in vivo.

One example of a situation where application of this medium may be desired arises when it is necessary to treat the oocytes collected from the subject with oocyte-maturation hormones in vitro due to the subject's intolerance of such hormones. The invention contemplates holding the oocytes in the maturation medium for a period of at least 24 hours, but no more than 60 hours, following collection of the oocytes, to promote development prior to fertilization.

The oocyte maturation medium may be comprised of one or more of NaCl, KCl, Mg₂SO₄, KH₂PO₄, Ca[lactate], NaHCO₃, amino acids and derivatives, a protein such as serum albumin, glucose, pyruvic acid and an antibiotic and includes a PDE inhibitor. The maturation medium may also include a ligand for inducing nuclear maturation of the oocyte, wherein the concentration of the ligand in the medium overcomes cAMP-induced meiotic arrest of the oocyte. However, as described above, it would be understood that the oocyte maturation medium need not contain the ligand, the ligand may be a separate component or part of a separate media.

The PDE inhibitor and ligand may be as hereinbefore described.

In one embodiment, the PDE inhibitor in the maturation medium is cilostamide. Generally, cilostamide is used at a concentration in the range from 0.01-100 μM and typically is in the range of 0.01-50 μM. For example in the bovine setting, a suitable concentration is between 10-30 μM, and in the human setting between 0.1-1.0 μM.

In a further specific embodiment, the ligand which induces nuclear maturation of the oocyte is FSH and/or EGF and the concentration of FSH and EGF is greater than 10 mlU/ml and 1 ng/ml, respectively and less than 500 mlU and 50 ng/ml, respectively, and preferably greater than 50 mlU and 5 ng/ml, respectively and less than 200 mlU and 20 ng/ml, respectively.

The components of the oocyte collection and maturation medium, and combination product thereof, of the present invention may be packaged separately in suitable containers (preferably sterilized) such as ampoules, bottles, or vials, either in multi-use or in unit forms. The containers may be hermetically sealed after being filled. The components may be in isolated form, or in purified or semi-purified form, and may contain additional additives for the stability and/or use of the components. Methods for packaging the various components are known in the art.

The collection and maturation media, and combination product thereof, of the present invention is suitable not only for use in humans, but also for culturing oocytes and embryos from other mammals. Thus, not only does the present invention have application for assisted reproduction technologies in humans, but it is also applicable to assisted reproduction techniques in non-human mammals, and other technologies of producing embryos in non-human mammals, such as the use of parthenogenic activation, somatic cell nuclear transfer and the use of totipotent stem cells.

In a sixth aspect the present invention provides a method of inducing oocyte maturation, the method comprising culturing an oocyte in a maturation medium comprising a phosphodiesterase (PDE) inhibitor and a ligand for inducing maturation of the oocyte, wherein the concentration of the ligand in the maturation medium overcomes cAMP-induced meiotic arrest of the oocyte, thereby maturing the oocyte.

The PDE inhibitor and the ligand, and concentrations thereof, according to this aspect of the invention may be as hereinbefore described. In one embodiment of this aspect of the invention, the phosphodiesterase inhibitor is cilostamide. In some embodiments, the ligand is FSH or EGF. Generally, the concentration of FSH is greater than 10 mlU/ml and the concentration of EGF is greater than 1 ng/ml.

In a seventh aspect, the present invention provides a method of inducing maturation of an oocyte which is in a state of meiotic arrest, the method comprising contacting the oocyte with a ligand at a concentration sufficient to overcome the meiotic arrest.

In one embodiment of this aspect of the invention the meiotic arrest is cAMP induced. The ligand, and concentration thereof, according to this aspect of the invention may be as hereinbefore described. In some embodiments, the ligand is FSH or EGF. Generally, the concentration of FSH is greater than 10 mlU/ml and the concentration of EGF is greater than 1 ng/ml.

In one embodiment of the second, sixth and seventh aspect of the invention, the method is part of an assisted reproduction technology. For example, the assisted reproduction technology comprises in vitro fertilisation.

Reference will now be made to the following experimental examples which embody the above general principles of the invention. However, it is to be understood that the following description is not to limit the generality of the above description. Thus, the invention encompasses any and all variations which become evident as a result of the teaching provided herein.

### Example 1

### Effect of cAMP Modulating Agents in Oocyte Collection and Maturation Media on Bovine Oocyte Maturation Kinetics

Oocyte quality plays an important role on embryonic development. For example, the inventors have shown that in vivo matured oocytes lead to higher blastocyst percentage than in vitro matured oocytes.

Unfortunately, current in vitro maturation techniques are suboptimal. In vivo, oocyte developmental competence is acquired gradually during the growth and development of the follicle. However, the inventors have shown that oocytes which have been retrieved from the follicle are capable of spontaneously overcoming meiotic arrest, thereby progressing to metaphase II before the cytoplasm has achieved full maturity.

Although immature oocytes can resume meiosis following isolation from the follicle, cytoplasmic maturation lags behind nuclear maturation. The inventors surmise that allowing more time for the immature oocyte to complete cytoplasmic maturation will improve oocyte developmental competence in vitro.

A possible strategy to improve developmental competence of oocytes is to keep them meiotically arrested in vitro for a prolonged period of time rather than allowing them to resume meiosis. Without wishing to be bound by theory, the inventors hypothesise that this delay gives the oocyte time to undergo cytoplasmic modifications (e.g. storage of mRNA and proteins, morphological changes, ultra structural remodeling) and might enhance synchronization of the starting population of immature oocytes to be used for downstream assisted reproduction applications.

In this regard, the inventors have tested the effect of including cAMP modulating agents in collection and handling (maturation) media on oocyte maturation kinetics such as intracellular cAMP levels, oocyte-cumulus cell gap junctional communications, nuclear maturation and embryonic development of cattle oocytes.

### Materials and Methods

Unless otherwise specified, all chemicals and reagents were purchased from Sigma (St. Louis, Mo., USA).

### Oocyte Collection and Selection—Pre-In Vitro Maturation (Pre-IVM) Phase

Bovine ovaries were collected from local abattoirs and transported to the laboratory in warm saline (30-35° C.). All ovaries collected on a day were pooled and used at random. Antral follicles (2 to 8 mm in diameter) were chosen for aspiration by using an 18-gauge needle and a 10 ml syringe.

Oocytes were aspirated with their cumulus vestments intact (COCs). A subset of the COCs were denuded of their cumulus vestment prior to assay (DO). Aspiration and subsequent selection procedures (pre-IVM phase) were performed for 2 hours where oocytes (COCs and DOs) were processed in various media (termed “pre-IVM phase media” or “pre-IVM media”). These included processing of oocytes either in follicular fluid, or in two types of collection medium. The collection medium for oocyte aspiration and selection included: (1) a bovine oocyte collection medium (termed “Bovine VitroMat”, Cook Australia, Eight Mile Plains, Old, Australia) supplemented with 50 μg/ml gentamycin and 0.2 mg/ml fatty acid-free bovine serum albumin (FAF-BSA; ICPbio Ltd, Auckland, NZ); or (2) the same medium supplemented with two cAMP modulators, namely an adenylate cyclase activator, forskolin (100 μM), and a non-specific PDE inhibitor, 3-isobutyl 1-methylxanthine (IBMX) (500 μM).

Millimolar stock concentrations of the cAMP modulators were stored at −20° C. dissolved in anhydrous dimethyl-sulphoxide (DMSO). Solutions containing modulators were diluted fresh for each experiment.

At the end of pre-IVM phase, intact COCs with compact cumulus vestments greater than five cell layers and evenly pigmented cytoplasm were selected under a dissecting microscope. Before in vitro maturation (IVM), COCs were washed twice in respective pre-IVM phase media, followed by two washes in IVM medium (see below).

### Oocyte In Vitro Maturation (IVM) Phase

The basic oocyte maturation medium (also referred to as the “IVM medium” or “IVM phase medium”) used for the IVM phase was a bovine maturation medium (termed Bovine VitroMat, Cook Australia), a medium formulated to closely replicate the ionic composition of bovine follicular fluid. Where indicated, the type-3 PDE specific inhibitor cilostamide (20 μM; Biomol Plymouth Meeting, Pa.) or the epidermal growth factor receptor (EGFR) kinase inhibitor, AG1478 (Alexis Biochemicals, San Diego, Calif.), were added to the IVM medium from a millimolar stock solution stored at −20° C. dissolved in DMSO. All IVM treatments were supplemented with 0.1 lU/ml follicle stimulating hormone (FSH) (Puregon, Organon, Oss, Netherlands). COCs were cultured in pre-equilibrated 300 μl drops overlaid with mineral oil and incubated at 39° C. with 5% CO₂ in humidified air.

### In Vitro Fertilization and Embryo Culture

At 24 or 30 hours after IVM, COCs were washed twice using Bovine VitroWash (Cook Australia), and transferred to insemination dishes containing in vitro fertilisation (IVF) medium (Bovine VitroFert, Cook Australia) supplemented with penicillamine (0.2 mM; Sigma), hypotaurine (0.1 mM; Sigma), and heparin (2 mg/ml; Sigma). Frozen semen from a single bull of proven fertility was used for insemination. Briefly, thawed semen was layered over a discontinuous (45%: 90%) Percoll gradient (Amersham Bioscience) and centrifuged for 20-25 minutes at 700 g. The supernatant was removed and the sperm pellet was washed with 500 μl Bovine VitroWash and centrifuged for a further 5 minutes at 200 g. Spermatozoa were resuspended with IVF medium (Bovine VitroFert), then added to the fertilization media drops (Bovine VitroFert, supplemented with 0.01 mM heparin, 0.2 mM penicillamine and 0.1 mM hypotaurine) at a final concentration of 1×10⁶ spermatozoa/ml. COCs were inseminated at a density of 10 μl of IVF medium per COC for 24 hours, at 39° C. in 6% CO₂ in humidified air. COCs were removed by gentle pipetting 23-24 hours post insemination and five presumptive zygotes were transferred into 20 μl drops of pre-equilibrated Cook Bovine VitroCleave medium (Cook Australia) and cultured under mineral oil at 38.5° C. in 7% O₂, 6% CO₂, balance N₂, for five days (day 1 to day 5). On Day 5, embryos in groups of 5-6 were transferred to 20 μl drops of pre-equilibrated Bovine VitroBlast (Cook Australia) at 38.5° C. overlaid with mineral oil and cultured to Day 8. Embryos were assessed for quality at Day 8 according to the definitions presented in Stingfellow and Seidel, 1998, Manual of the International Embryo Transfer Society. In. (IETS: Savoy, Ill., USA) and were performed independently and blinded by an experienced bovine embryologist.

### Blastocyst Differential Staining

Blastocysts were placed into 0.5% pronase at 37° C. to remove the zona, followed by a brief wash in 4 mg/ml poly-vinyl alcohol (PVA) in phosphate-buffered saline (PBS/PVA). Zona-free blastocysts were then incubated in 10 mM trinitrobenzene sulfonic acid in PBS/PVA at 4° C. for 10 minutes. Blastocysts were subsequently incubated with 0.1 mg/ml anti-dinitrophenol-BSA antibody (Molecular Probes, Eugene, Oreg., USA) at 37° C. for 10 minutes, and then placed in guinea pig serum with propodium iodide for 5 minutes at 37° C. Blastocysts were washed and incubated in 10 μg/ml propidium iodide for 20 minutes at 37° C. (to stain the trophectoderm), followed by 4 μg/ml bisbenzimide (Hoechst 33342; Sigma-Aldrich) in 100% ethanol at 4° C. overnight (to stain both the inner cell mass (ICM) and trophectoderm). Blastocysts were then whole mounted in a drop of 80% glycerol in PBS on microscope slides and coverslips were sealed with nail polish. Blastocysts were then examined under a fluorescence microscope (Olympus, Tokyo, Japan) at 400× equipped with an ultraviolet filter and a digital camera attached to determine total and compartment cell counts where inner cell mass (ICM) nuclei appeared blue and trophectoderm (TE) nuclei stained pink.

### Measurement of Intracellular Camp

Cyclic AMP content of COCs and denuded oocytes (DOs), i.e. those oocytes cultured as COCs but denuded of their cumulus vestment prior to assay, were measured using a radioimmunoassay method described and validated previously (Reddoch et al., 1986, Endocrinology 119: 879-886). After time end-points, 6-10 COCs and 21-24 DOs were washed in VitroCollect (Cook Australia), transferred to 0.5 ml of ethanol (100%) and stored at −20° C. Before cAMP measurements, samples were vortex agitated for 30 seconds and then centrifuged at 3000 g for 15 minutes at 4° C. Briefly, supernatants were collected, evaporated, resuspended in assay buffer (50 mM sodium acetate, pH 5.5) and acetylated by the addition of triethylamine (AJAX Chemicals, Sydney, Australia) and acetic anhydride (BDH Laboratory Supplies, Poole, England) 2:1 v/v. cAMP was measured in duplicates after appropriate dilution. ¹²⁵I-labelled cAMP (specific activity of 2175 Ci/mM) and cAMP antibody (as prepared by Reddoch et al supra) were added to samples and left overnight at 4° C. The following day, 1 ml cold 100% ethanol was added and the samples were centrifuged at 3000 g. The supernatant was removed and the pellet dried and counted using a gamma counter. Duplicates of known concentration samples were used to produce a standard curve (4-1024 fmol cAMP).

### Oocyte Nuclear Morphology Assessment

At the end of the incubation, COCs were denuded and oocytes were fixed for 30 min in 4% paraformaldehyde in PBS (pH 7.4). Oocytes were then permeabilized in 0.1% Triton X-100 in 0.1% sodium citrate for 1 hour, then transferred to 0.001% 4′,6-diamidino-2-phenylindole (DAPI), a fluorescence stain for nuclear material, for 15 min. Oocytes were rinsed 3 times in PBS+0.03% BSA, mounted on slides and evaluated for nuclear status at 400× using an Olympus fluorescence microscope. Stages of germinal vesicle (GV) and subsequent meiotic development were assessed based on known techniques (Chohan and Hunter, 2003, Anim. Reprod. Sci. 76: 43-51). In brief, GV chromatin of bovine oocytes was classified into: GV I—condensed filamentous chromatin around the nucleolus and nuclear membrane; GV II—filamentous chromatin surrounding the nucleolus; GV III—filamentous chromatin clumps are distributed in the nucleus and the nucleolus has disappeared; GV IV—chromatin has condensed into a thick clump; Early diakenesis-chromatin beginning to condense into a single lump; Diakenesis-chromatin has condensed into a single lump; Metaphase I—tetrads are aligned on the spindle; and Metaphase II—the metaphase chromatin is evident as well as a small chromatin-containing polar body.

### Oocyte-Cumulus Cell Gap Junctional Communication Assay

Cumulus-oocyte gap junctional communication (GJC) was measured by quantitative fluorescence microscopy of calcein transfer to the oocyte as described previously (Thomas et al., 2004, In “Biol. Reprod.”, pp 1142-1149). GJC was measured either after 2 hours of pre-IVM phase or followed by an additional 3 hours of IVM with or without cilostamide (20 μM). A mean number of 10-12 oocytes were used in each treatment group in each of four replicate experiments. After culture, COCs were transferred to a solution of 1 μM calcein-AM (3′,6′-di(O-acetyl)-2′,7′-bis[N,N-bis(carboxymethyl)amino methyl]-fluorescein, tetraacetoxy methyl ester; C-3100; Molecular Probes; Eugene, Oreg., USA) freshly prepared in a BSA-free Bovine VitroCollect (Cook Australia) supplemented with polyvinyl alcohol (PVA; 0.3 mg/ml). COCs were cultured with the dye for 15 minutes, and then unincorporated dye was removed by three washes in calcein-AM-free Bovine VitroCollect (Cook Australia) and incubated for a further 25 minutes to allow transfer from the cumulus cells to the oocyte. Prior to fluorescence microphotometry, COCs were completely denuded of their surrounding cumulus cells using vigorous pipetting so that only dye confined within the denuded oocyte after transport via gap junctions was measured. Within 30 minutes of denuding, the intra-oocyte fluorescence emission of calcein in oocytes was measured using a fluorophotometric-inverted microscope (Leica, Wetzlar, Germany).

### Statistical Analysis

Statistical analyses were conducted using Prism 5.00 GraphPad for Windows (GraphPad Software, San Diego, Calif., USA). Statistical significance was assessed by ANOVA followed by either Dunnett's or Bonferroni's multiple-comparison post-hoc tests to identify individual differences between means. All values are presented with their corresponding standard error of the mean (SEM).

### Results

cAMP Content of Bovine COCs and Dos During Pre-IVM Phase

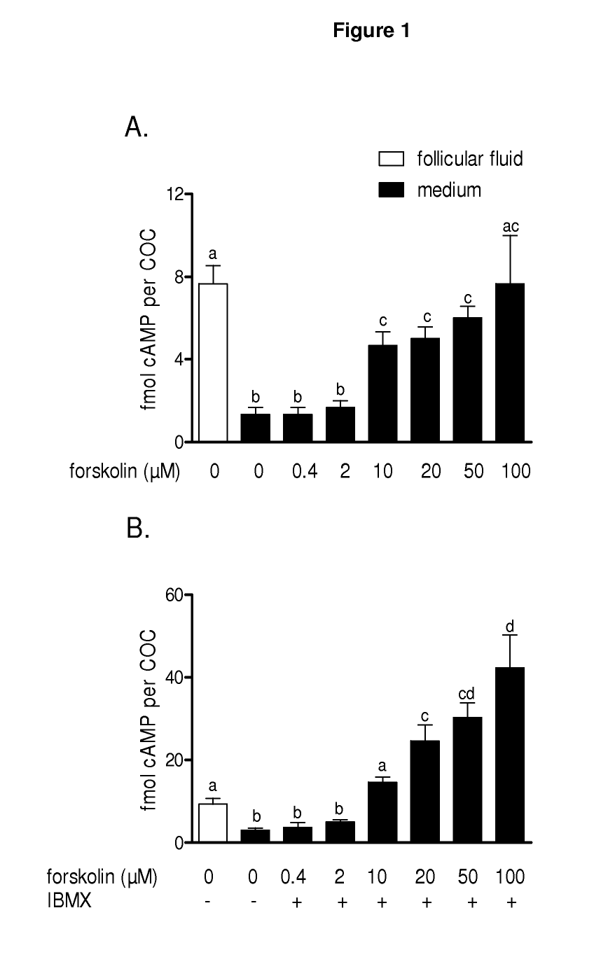

FIGS. 1 to 4 show the effect of various pre-in vitro maturation (pre-IVM) phase treatments over time on the cAMP content of COCs and oocytes. As shown in FIG. 1A, after 2 hours of pre-IVM, forskolin significantly elevated (P<0.05) cAMP levels within COCs in a dose dependent manner. Compared to the follicular fluid control, only the highest concentration of forskolin (100 μM) gave a similar value. However, neither 0.4 μM, nor 2 μM forskolin showed any increase in cAMP levels, which was not significantly different from the control treatment (collection medium without cAMP modulators). In combination with IBMX, increasing concentrations of forskolin induced a 20-fold increase in cAMP in a dose dependent manner over the levels observed in absence of IBMX and presence of forskolin alone (FIG. 1B). Hence, forskolin or IBMX alone maintains but does not elevate cAMP levels substantially higher than the controls.

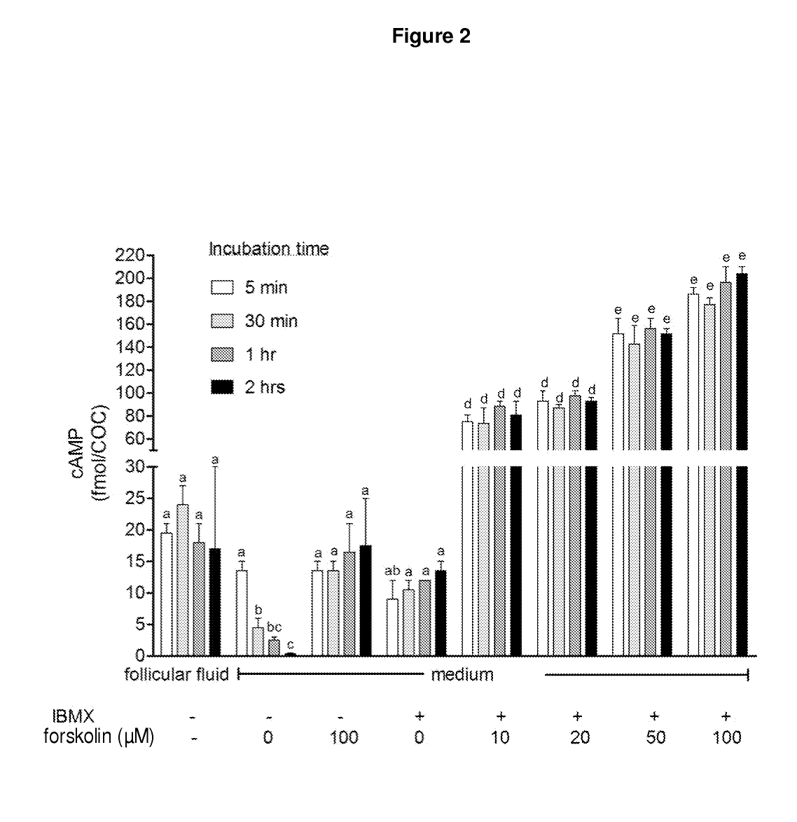

The aim of the experiment in FIG. 2 was to examine the effect of pre-IVM duration when COCs are incubated with IBMX and increasing doses of forskolin (10-100 μM) on COC cAMP levels. As shown in FIG. 2, COC cAMP levels were maintained during the pre-IVM period among all treatment groups except the control group (collection medium without cAMP modulators). When COCs were incubated in collection medium alone, cAMP levels dropped significantly (P<0.05) after 30 minutes from 14 to 4 fmol/COC, and continued to drop significantly with increasing time (0.4 fmol/COC) up to 2 hours of incubation. Treating COCs with IBMX or forskolin alone maintained cAMP levels for 2 hours. However, a dramatic induction of up to 20 fold in cAMP levels was noted when COCs were incubated in the presence of IBMX and increasing doses of forskolin, and this increase in cAMP levels was notable regardless of incubation period. The highest cAMP levels were noted when COCs were incubated in the presence of IBMX and 50 or 100 μM forskolin (approximately 165 fmol/COC). There was no effect of time in increasing cAMP in COC except in the control group (no cAMP modulators).

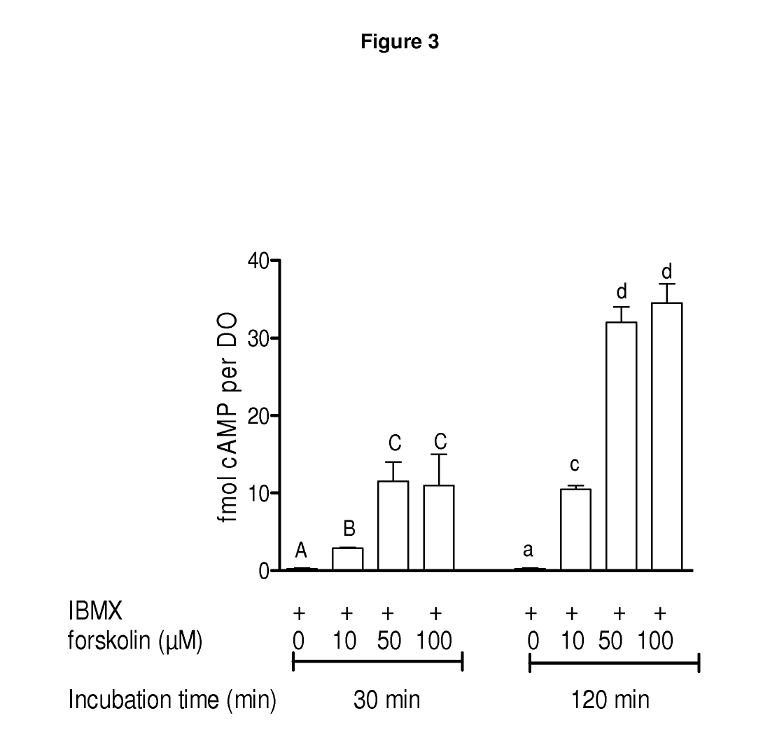

The aim of the experiment in FIG. 3 was to examine cAMP levels in the oocyte (COCs denuded after pre-IVM) and how these levels change in the presence of cAMP modulators with extending the incubation time in the pre-IVM phase. As shown in FIG. 3, after 30 minutes of incubation, cAMP levels were significantly higher (12 fmol/oocyte) when COCs were incubated in the presence of IBMX and 10, 50 or 100 μM forskolin, compared to control (0.5 fmol/oocyte, P<0.05). After 2 hours, levels of cAMP are further significantly increased up to 34 fmol/oocyte, compared to control (0.1 fmol/oocyte, P<0.05). It appears that increasing the incubation time in the pre-IVM phase, leads gradually to a substantial increase in intra-oocyte cAMP in the presence of IBMX and high concentrations of forskolin.

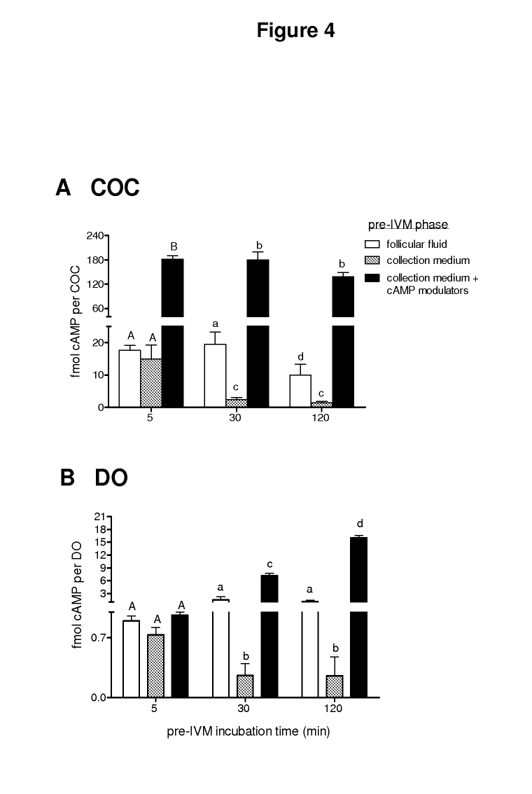

FIG. 4 shows the effect of various pre-in vitro maturation (pre-IVM) phase treatments over time on the cAMP content of intact COCs and within oocytes: (A) oocytes collected and assayed with their cumulus vestments intact (COC); and (B) oocytes collected as COCs but denuded of their cumulus vestment prior to assay (DO). Oocytes of the two types were collected and selected in pure follicular fluid, collection medium, or collection medium supplemented with cAMP modulators (forskolin and IBMX). Values are expressed as the mean concentration of cAMP per oocyte or complex±SEM of three replicates using 6-10 COCs or 21-24 DOs per treatment replicate. Means within the same graph/oocyte type with different letters (A, a, B, b or c) indicate significantly different amounts of cAMP between individual treatments or end time points (two-way ANOVA, P<0.05).

As seen in FIG. 4A, after 5 min of pre-IVM phase, the cAMP level in COCs collected in the presence of cAMP modulators was 9 fold higher than the control groups, i.e. absence of cAMP modulators or pure follicular fluid (P<0.0001). The increase in cAMP level was tuned during (30 min), and at the end of (2 hours), pre-IVM phase. Follicular fluid maintained the level of cAMP in COCs for 30 min; however, the level dropped at the end of pre-IVM phase (20±3 to 10±3 fmol/COC) (P<0.05). For those COCs collected in the absence of cAMP modulators, the cAMP level dropped sharply (from 15±4 to 2±1 fmol/COC) (P<0.05) shortly after isolation of the oocyte from the follicle (30 minute time-period).

Denuded oocytes (DOs) were used to evaluate the effect of the surrounding cumulus cells on intra-oocyte cAMP levels (FIG. 4B). Intra-oocyte cAMP levels were approximately 0.9±0.1 in all treatments 5 minutes post isolation from the follicle. After 30 minutes of processing in the pre-IVM phase media, the cAMP level in oocytes treated with cAMP modulators was significantly higher than the control groups and increased with incubation time (16±0.1) through to the end of pre-IVM phase (2 hours). Follicular fluid maintained the intra-oocyte cAMP level during the whole pre-IVM period, whereas for oocytes collected in the absence of cAMP modulators, the intracellular cAMP level dropped significantly within 30 minutes and continued to drop until the end of the pre-IVM phase (0.3±0.2)(P<0.05).

Effect of cAMP Modulating Agents in Pre-IVM Phase and Type-3 PDE Inhibition on Spontaneous Oocyte Maturation (GV/GVBD)

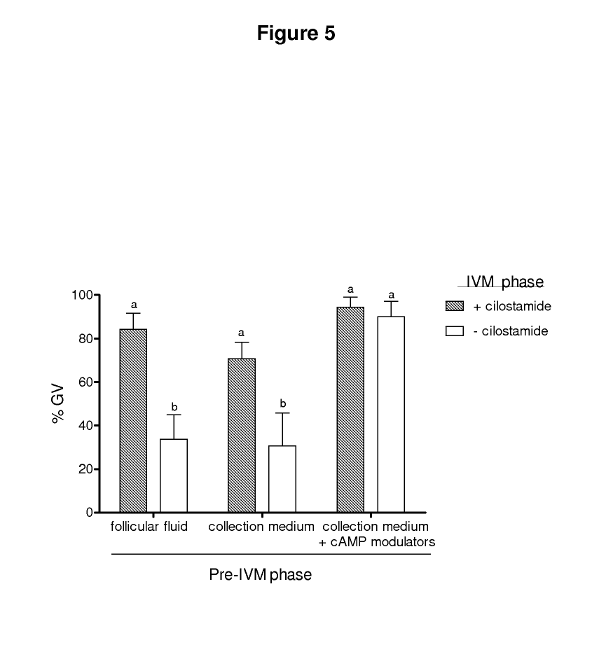

FIG. 5 shows the effect on spontaneous oocyte maturation (GV/GVBD) following incubation of cumulus-oocyte complexes in various pre-in vitro maturation (pre-IVM) and IVM phase media.

Bovine COCs were incubated for 2 hours in the pre-IVM phase media as hereinbefore described, followed by 7 hours culture in the presence of FSH, and in the presence or absence of cilostamide (20 μM). Oocytes were then fixed and assessed for meiotic progression and classified as GV (germinal vesical intact—still under meiotic arrest) or GVBD (germinal vesical breakdown—resumption of meiosis). A mean number of 45 oocytes were used in each treatment group and time-point from four replicate experiments. The presence of letters (a or b) above each column indicate a statistical difference between the means as determined by ANOVA analysis followed by Bonferronni's post hoc test, P<0.05.

As seen in FIG. 5, inclusion of FSH and cilostamide in the culture media during IVM phase significantly lowered the rates of GVBD among all pre-IVM treatments compared to controls (cilostamide (−) in IVM). However, in the absence of cilostamide in the media, the majority of oocytes did commence GVBD, except those oocytes processed in collection medium which included cAMP modulators during pre-IVM, where there was a significant delay in GVBD (P<0.05). These results led the inventors to investigate GV configuration changes at the end of the pre-IVM phase and during the IVM phase (see below).

### Effect of Camp Modulating Agents in Pre-IVM Phase and Type-3 PDE Inhibition on Oocyte Germinal Vesicle Configurations

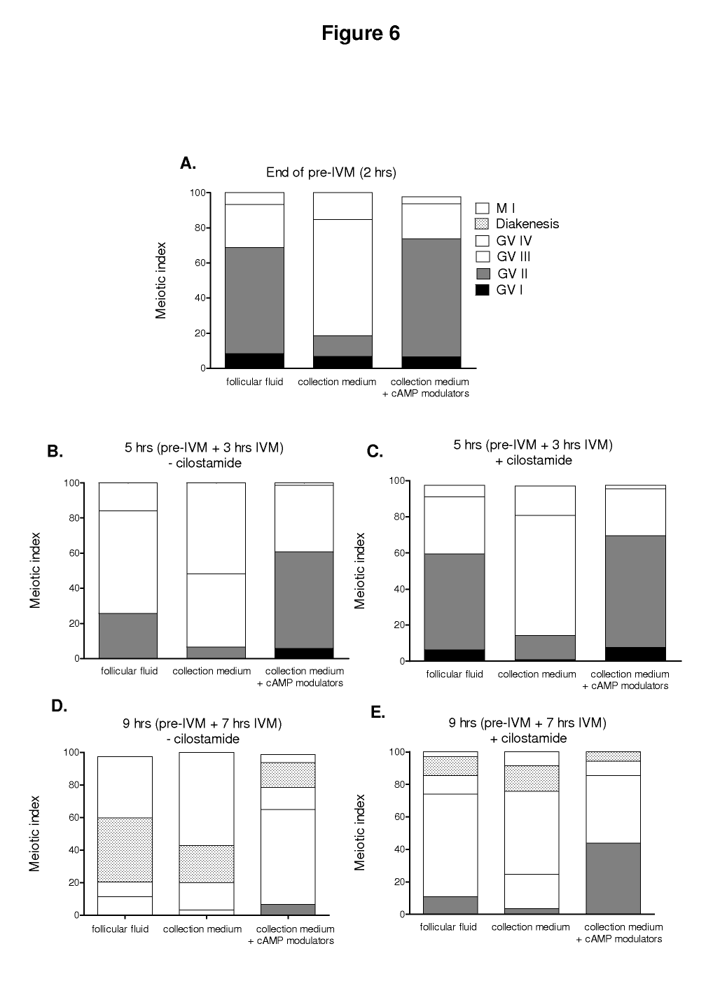

FIG. 6 shows the effect on germinal vesicle (GV) configurations of cumulus-oocyte complexes cultured in various pre-IVM and IVM phase media. Oocytes were exposed to pre-IVM phase media comprising pure follicular fluid, collection medium, or collection medium supplemented with cAMP modulators (forskolin and IBMX) (A), followed by extended culture in the presence (C; E) or absence (B; D) of the type-3 PDE inhibitor, cilostamide (20 μM), plus follicle stimulating hormone. Oocytes were then fixed and assessed for GV configuration at 2, 5 and 9 hours. A mean number of 40 oocytes were used in each treatment group and time-point from four replicate experiments.

As seen in FIG. 6A, at the end of pre-IVM phase (2 hours), the majority of the oocytes cultured in follicular fluid (61%±5) or cultured in collection medium supplemented with cAMP modulators (67%±5) (P<0.05) were at the GV II stage. However, oocytes processed in collection medium alone had a decreased GV II percentage (P<0.05) and had progressed to GV III (66%±5) (P<0.001).

After 5 hours (2 hours of pre-IVM and 3 hours of IVM+cilostamide) collected oocytes were already arrested at their destined GV stages prior to the IVM incubation (FIG. 6C). When compared to FIG. 6B, this indicates that the presence of FSH and cilostamide in the culture media during the first hours of IVM incubation prevented COCs from progressing through their GV configurations compared to COCs cultured in the presence of FSH alone (IVM-cilostamide). As seen in FIG. 6B, oocytes processed in pure follicular fluid were progressing to GV III (58%±8), whereas most of the oocytes which were processed in culture media in the absence of cAMP modulators were at GV III (41%±10) or at GV IV (52%±12) (P<0.05). Surprisingly, even in the absence of cilostamide in the culture media during IVM, COCs were still arrested at GV II (55%±3) when processed with cAMP modulators (FIG. 6B, right column).

As seen in FIG. 6E, right column, after 9 hours of oocyte culture (2 hours of pre-IVM and 7 hours of IVM+cilostamide), COCs processed in media comprising cAMP modulators were progressed in maturation with the majority of COCs at GV II (44±3%) or at GV III (42±3%). This was compared to oocytes which were selected in pure follicular fluid where the majority of oocytes were at GV III (63±6), and oocytes which were processed in collection medium lacking cAMP modulators where the oocytes had progressed to GV IV (51%±10)(P<0.05)(FIG. 6E, left and middle columns respectively). After 9 hours and in the presence of FSH alone (IVM-cilostamide), most oocytes which were treated with cAMP modulators had progressed to the GV III stage (58%±4) (FIG. 6D, right column), whereas the majority of oocytes which were processed in pure follicular fluid progressed to diakenesis (40%±4) and M I (38%±4)(FIG. 6D, left column). In contrast, of those oocytes processed with collection medium lacking cAMP modulators, 23%±6 were at the diakenesis stage and 57%±5 had progressed to the M I stage (P<0.05) (FIG. 6D, middle column).

Effect of cAMP Modulating Agents During Pre-IVM and IVM on Oocyte-Cumulus Cell Gap Junctional Communication

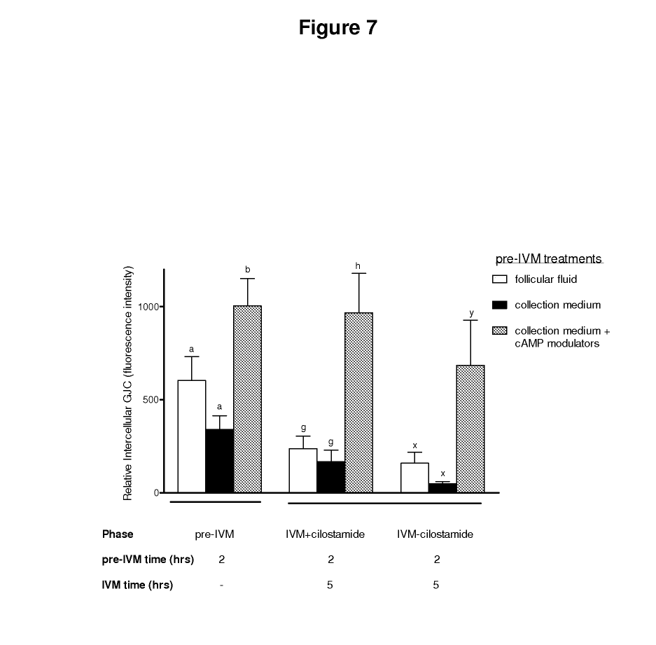

The oocyte-cumulus gap junctional communication (GJC) assay was performed at the end of pre-IVM phase and at the end of 5 hours of oocytes culture (2 hours of pre-IVM and 3 hours of IVM±cilostamide). As seen in the columns 1 to 3 of FIG. 7, at the end of pre-IVM phase (2 hours), levels of gap junctional communication between the oocyte and the cumulus cells significantly decreased from a fluorescence intensity of approximately 1000 (when the oocytes were aspirated and processed in collection medium supplemented with cAMP modulators) to approximately 400 and 600 (when the oocytes were aspirated and processed in follicular fluid, or collection medium without cAMP modulators, respectively)(P<0.05).

Moreover, levels of gap junctional communication dramatically decreased after 3 hours of IVM culture (post pre-IVM phase) when COCs were collected either in pure follicular fluid, or collection medium without cAMP modulators. The exception were COCs which were processed in collection media supplemented with cAMP modulators during pre-IVM phase, regardless of the presence or absence of cilostamide during IVM (P<0.05) (FIG. 7, columns 4-9). The inclusion of FSH (and the presence or absence of cilostamide) in the culture medium had no effect in maintaining the level of gap junctional communication between the oocyte and its surrounding cumulus cells.

The presence of letters (a, b, g, h or x) above each column in FIG. 7 indicate a statistical difference between the means as determined by ANOVA analysis followed by Bonferronni's post hoc test.

### Effect of Camp Modulating Agents in Pre-IVM Phase and Type-3 PDE Inhibition on Oocyte Meiotic Progression to MII Stage

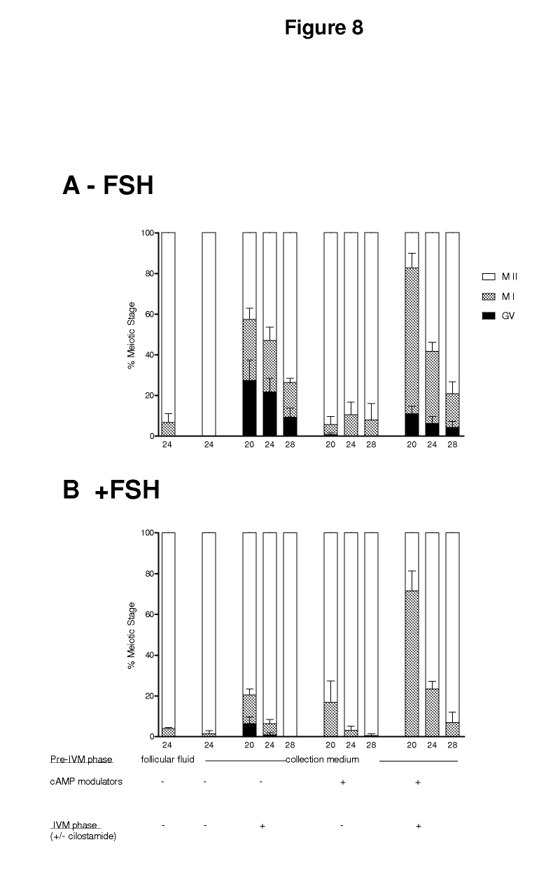

FIG. 8 shows the effect of follicle stimulating hormone (FSH) on inducing meiotic maturation of cumulus-oocyte complexes (COCs) exposed to various pre-IVM and IVM phase media. As indicated above, oocytes were aspirated and selected in either follicular fluid, collection medium or collection medium supplemented with 100 μM forskolin (FSK) and 500 μM IBMX for 2 hours. COCs were then cultured in 2 media (+/−cilostamide) in the absence of FSH (A) or presence of FSH (B). Oocytes were then fixed and assessed for meiotic progression at 20, 24 and 28 hours. A mean number of 45 oocytes were used in each treatment group and time-point from four replicate experiments.

As seen in FIG. 8A, in the absence of FSH, cilostamide treatment delays oocyte meiotic progression to M II stage (50%) at 24 hours of IVM either in the presence or absence of cAMP modulators in the pre-IVM media. However at 20 hours of IVM, 83%±7 of the oocytes were at M I stage when the oocytes were processed in collection medium supplemented with cAMP modulators in the pre-IVM phase, compared with 36%±4 of oocytes which were at M I stage when the oocytes were processed in collection medium lacking cAMP modulators (FIG. 8A, columns 9 and 3 respectively).

As seen in FIG. 8B, in the presence of FSH, the inhibitory effect of cilostamide in delaying oocyte progression to the M II stage was overridden by FSH present in the IVM media. For example, while GV stage oocytes were detected in the presence of cilostamide but absence of FSH (FIG. 8A), such oocytes were not detected when FSH is present in the IVM media. Interestingly, after 20 hours of IVM+cilostamide treatment, 72%±5 of oocytes were at M I stage when they were processed in collection medium supplemented with cAMP modulators during the pre-IVM phase (FIG. 8B, column 9) compared to 21%±4 of oocytes when they were processed in collection medium alone (FIG. 8B, column 3).

Intracellular Concentration of cAMP in Oocytes after Pre-IVM and IVM Phases

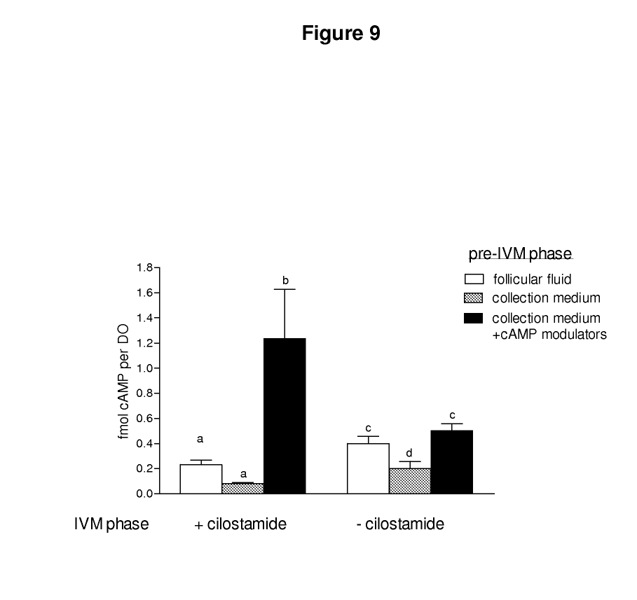

FIG. 9 shows the intracellular cAMP concentration of oocytes exposed to various pre-IVM and IVM phase media. Oocytes were first denuded of their cumulus vestment prior to the assay (DOs), and were then collected and handled in 3 different pre-IVM phase media (follicular fluid, collection medium alone, and collection medium supplemented with 100 μM forskolin (FSK) and 500 μM IBMX), followed by extended culture in the presence or absence of the type-3 PDE inhibitor cilostamide (20 μM) for 24 hours. The data represent the mean cAMP level per DO±SEM of 4 replicates. Each measurement was conducted on 24 DO. The presence of letters (a, b, c or d) above each column indicate a statistical difference between the means as determined by ANOVA analysis followed by Dunnett's post hoc test.

As seen in FIG. 9, after 24 hours of culture, the intracellular concentration of cAMP in DOs remained significantly higher (up to 15 fold)(P<0.05) when the oocytes were collected and processed in collection medium supplemented with cAMP modulators during pre-IVM (as opposed to those cultured in pure follicular fluid, or collection medium lacking cAMP modulators), provided cilostamide was also present in the culture media.

### Effect of an Epidermal Growth Factor Receptor Kinase Inhibitor on FSH-Induced Oocyte Maturation in the Presence of a Type-3 PDE Inhibitor (Cilostamide)

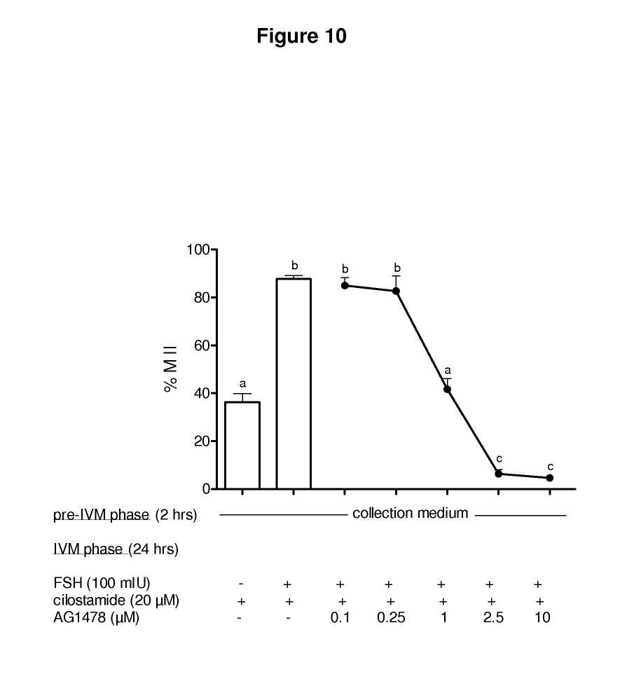

As shown in FIG. 10, the inhibitory effect of cilostamide on oocyte meiotic progression was overridden by the addition of FSH in culture. It was therefore an interest to examine if the FSH-induced maturation (meiotic induction) was suppressed by antisera to the Epidermal Growth Factor Receptor (EGFR), for example by examining if the EGFR kinase inhibitor, AG1478, could affect this pattern of maturation.

In this regard, FIG. 10 shows the effect of an epidermal growth factor receptor (EGFR) inhibitor on follicle stimulating hormone (FSH)-induced oocyte maturation in the presence of the FSH (100 mlU), the PDE inhibitor cilostamide (20 μM), and increasing doses of the EGFR inhibitor AG1478. Oocytes were then fixed and assessed for meiotic progression at 24 hours. A mean number of 40 oocytes were used in each treatment group and time-point from three replicate experiments. The presence of letters (a, b or c) above each column or data point indicate a statistical difference between the means as determined by ANOVA analysis followed by Dunnett's post hoc test.

As seen in FIG. 10, the addition of increasing doses of AG1478 in the IVM media significantly reduced the maturation percentage (from >80% to approximately 5%)(P<0.05), thereby completely suppressing the induction response. This indicates that EGF signalling is required in FSH-induced oocyte maturation.

Effect of cAMP Modulating Agents During Pre-IVM and IVM Phases on Cleavage and Development to the Blastocyst Stage

As shown in Table 1, inclusion of cAMP modulators in collection media during the pre-IVM phase improves cleavage rates compared to absence of the modulators (89±2.0% vs. 78±2%, respectively, P<0.05), and improves blastocyst development (32±3% vs. 26±3%, P<0.05) when oocytes are matured in standard IVM medium in the presence of FSH for 24 hours.

TABLE 1

| | Number | % Cleavage | % Blastocysts |
|---|---|---|---|
| Pre-IVM Treatments | of oocytes | rate | from cleaved |
| Follicular fluid | 186 | 89.5 ± 1.4^{a} | 38.5 ± 5.7^{a} |
| Collection medium | 205 | 77.8 ± 3.6^{b} | 26.5 ± 4.0^{b} |
| Collection medium + | 200 | 88.5 ± 1.9^{a} | 32.0 ± 2.1^{ab} |
| cAMP modulators | | | |

Note:
^{a,b}Values with different superscripts within the same column represent a statistically significant difference (P < 0.05). Values are expressed as (mean ± SEM).

As previously shown in FIG. 8, pre-treating oocytes with cAMP modulators during the pre-IVM phase, and inclusion of cilostamide in the IVM medium, delays the onset of M II in oocytes by 4 hours in the combined presence of FSH. Therefore, cumulus-oocyte complexes (COCs) were aspirated and selected in either follicular fluid, collection medium or collection medium supplemented with 100 μM forskolin (FSK) and 500 μM IBMX for 2 hours. The COCs were then matured by the addition of FSH for 24 or 30 hours in the presence of cilostamide (20 μM). Oocyte developmental capacity was then assessed after in vitro fertilization and embryo development was assessed by the cleavage rate and blastocyst rate on day 8. A mean number of 45 oocytes were used in each treatment group from four replicate experiments. The presence of letters (a, b, x, y or z) above each column indicate a statistical difference between the means as determined by ANOVA analysis followed by Bonferronni's post hoc test. Table 2 provides the results of this study with respect to percent blastocyst formation.

TABLE 2

| | | Maturation | % |
|---|---|---|---|
| Collection Phase | IVM | time | Blastocysts |
| CM | control | 24 | 27% |
| CM | +cilostamide | 24 | 22% |
| CM | +cilostamide | 30 | 29% |
| Follicular Fluid | control | 24 | 39% |
| Follicular Fluid | +cilostamide | 24 | 38% |
| Follicular Fluid | +cilostamide | 30 | 61% |
| CM + cAMP modulators | control | 24 | 32% |
| CM + cAMP modulators | +cilostamide | 24 | 48% |
| CM + cAMP modulators | +cilostamide | 30 | 69% |

Note:
CM—Collection medium

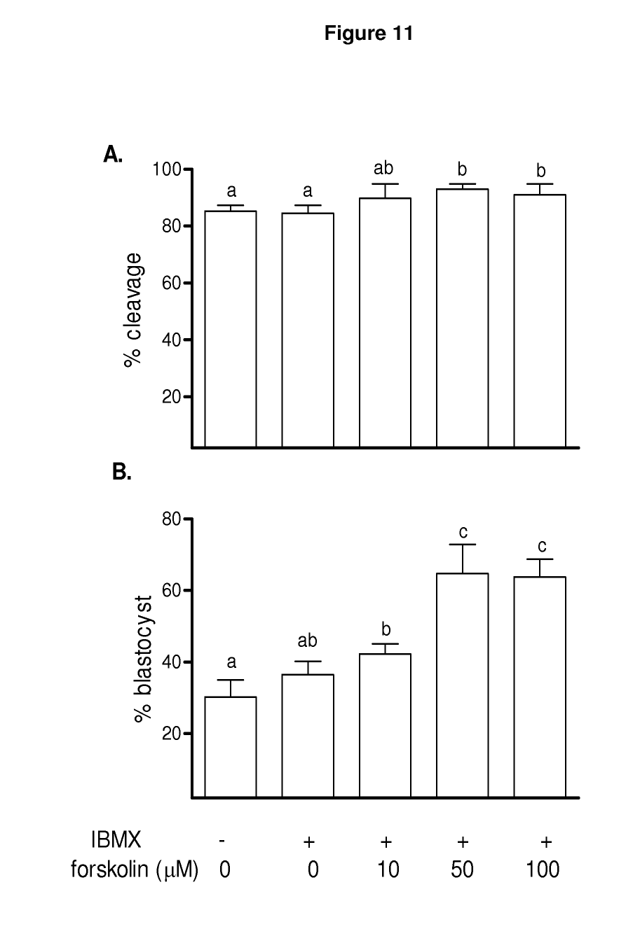

FIG. 11A shows that the cleavage rates of oocytes incubated in the presence of IBMX and 50 or 100 μM forskolin for 30-60 minutes prior to IVM were significantly greater than that of oocytes incubated in the presence of IBMX alone or control treatment (P<0.05). Development to the blastocyst stage (FIG. 11B) was similarly improved for oocytes incubated in the presence of IBMX and 50 or 100 μM forskolin for 30-60 minutes prior to IVM compared to control treatment (P<0.05). Moreover, the rate of development to the blastocyst stage was not significantly different between IBMX alone or IBMX and 10 μM forskolin group, however were significantly higher than control (no cAMP modulators) (P<0.05). Hence, increasing cAMP levels within the oocyte prior to IVM leads to a substantial improvement in developmental outcomes when COCs are matured in the presence of cilostamide.

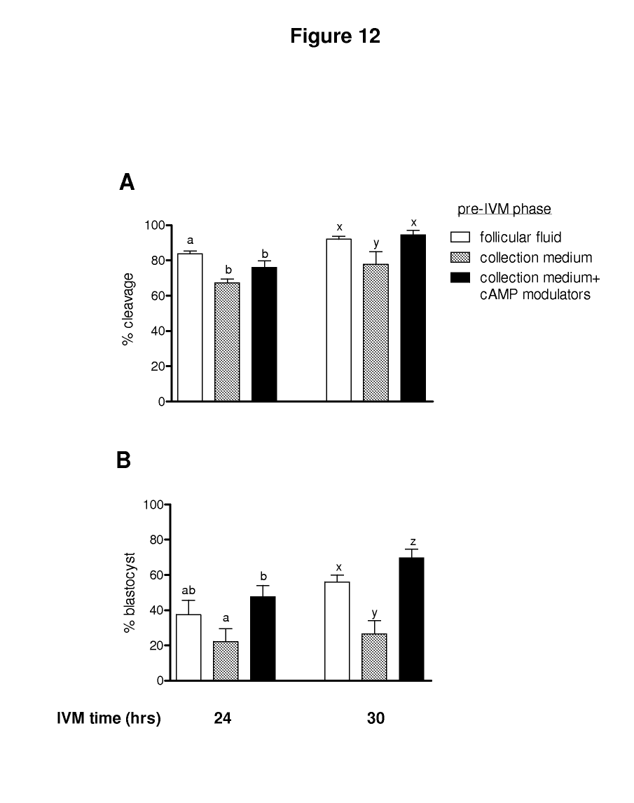

As seen in FIG. 12A, at 24 hours, the cleavage rate for oocytes pre-treated either in follicular fluid or collection medium supplemented with cAMP modulators was approximately 80%, which was significantly higher than oocytes which were collected in the absence of cAMP modulating agents during pre-IVM phase (67%)(P<0.05). Moreover, as seen in FIG. 12B the rate of development to the blastocyst stage was also significantly higher (48%) in the presence of cAMP modulating agents in the pre-IVM media compared to their absence (22%).

However, oocytes fertilised at 30 hours yielded a dramatic increase in blastocysts when pre-treated with cAMP modulating agents and matured with FSH+cilostamide (increase from 42% to 69% blastocysts)(FIG. 12B, columns 3 and 6 respectively) compared to oocytes collected and handled in the absence of cAMP modulating agents (27% blastocysts)(P<0.05)(FIG. 12B, column 5).

Effect of cAMP Modulating Agents During Pre-IVM and IVM on Blastocyst Cell Numbers

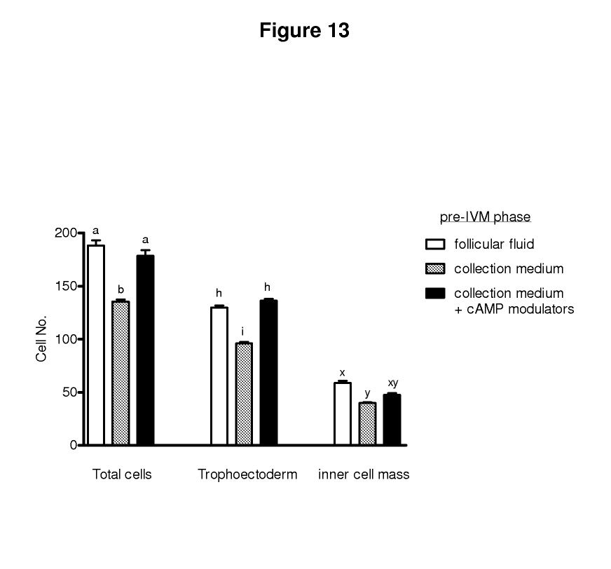

FIG. 13 shows the effect of the presence of cAMP modulating agents present in various pre-IVM and IVM phase media on blastocyst cell numbers. Cumulus-oocyte complexes (COCs) were aspirated and selected in either follicular fluid, collection medium or collection medium supplemented with 100 μM forskolin (FSK) and 500 μM IBMX for 2 hours. The COCs were then matured by the addition of FSH for 30 hours in the presence of cilostamide (20 μM). The number of total, inner cell mass and trophectoderm cells (mean±SEM) of Day 8 expanded and hatched blastocysts was determined. A mean number of 20-30 expanded or hatched blastocyst were used in each treatment group. The presence of letters (a, b, h, l, x or y) above each column indicate a statistical difference between the means as determined by ANOVA analysis followed by Bonferronni's post hoc test.

As seen in FIG. 13, in the presence of cilostamide in the IVM phase media, oocytes processed in pure follicular fluid or in the presence of cAMP modulators in the pre-IVM phase media significantly increased blastocyst total cells, trophoectoderm and inner cell mass numbers compared to oocytes processed in culture media lacking cAMP modulators in the pre-IVM phase (P<0.05).

The current study was undertaken to test the hypothesis that developing a process for inducing oocyte maturation in vitro by manipulating intracellular cAMP levels in the oocyte immediately following collection and during the maturation process, synchronizes nuclear and cytoplasmic compartments, thereby leading to improved developmental competence of maturing oocytes.

The participation of cAMP in controlling oocyte maturation in mammals has been shown in the past few years. Nevertheless, the molecular mechanisms of the series of coordinate events guided by cAMP have not been clearly defined. The experimental data as hereinbefore described showed that the efficacy of PDE inhibitors may be enhanced by modulating cAMP levels during the period preceding IVM. In the first part of this study (FIGS. 1 to 4), results obtained from measuring cAMP levels in whole COCs or DOs were consistent with previously measured data in cows at the time of follicle removal. In particular, results from the present experiments showed that the intracellular cAMP concentration during the interval between oocyte isolation from the follicle and the beginning of in vitro maturation (IVM) appears critical for oocyte kinetics and for the achievement of higher developmental competence. Indeed, modulating oocyte cAMP levels in the pre-IVM phase appeared to have a long effect in maintaining high oocyte cAMP levels after 24 hours of IVM in the presence of cilostamide in culture (FIG. 9).

The different collection conditions used in this study have been found to exert a profound effect on oocyte intracellular cAMP levels, oocyte meiotic progression, oocyte-cumulus cells gap junctional communication and finally oocyte developmental competence. The present results demonstrated that modulating cAMP for a short period of time from the time of COC isolation can lead to sustained gap junctional communication between cumulus cells and oocyte (FIG. 7).

The increase in gap junctional communication observed in this study, when cAMP levels were modulated during the pre-IVM phase, was due to prolonged gap junction conductance and/or the prevention of gap junction removal from the cumulus cell-derived meiosis-modulating factors to the oocyte, thereby delaying GV configurations or GVBD after 7 hours of oocyte culture (FIGS. 5 and 6).

An intriguing question was how the exogenous modulation of cAMP can evoke a stimulatory or inhibitory response in germinal and somatic compartments. In the described experiments, the intra-oocyte cAMP level was 15 fold higher when cAMP modulators were present in culture media during the pre-IVM and IVM phases compared to other treatments (FIG. 9). This had further been supported from the experiments testing the EGFR kinase inhibitor, AG1478, during IVM (FIG. 10). Previous studies have shown that the epidermal growth factor (EGF) can be produced by ovarian cells, and is the most potent stimulator of both cumulus expansion and meiotic resumption amongst a number of other growth-promoting factors. It has also been shown that EGF-like peptides can mediate the actions of gonadotrophins in pre-ovulatory follicles. EGF-like peptides therefore can play an intimate role in the paracrine signalling between metabolic pathways of different cell types leading to oocyte maturation and follicular rupture.

Therefore, the aim of one of the experiments, the results of which are shown in FIG. 10, was to examine the role of EGF-like peptides in follicle-stimulating hormone-induced maturation of cilostamide-cultured COCs. As mentioned previously, an increase in intra-oocyte cAMP level up to 15 fold delays meiotic progression to M II up to 28 hours (FIG. 8). Interestingly, the inhibitory effect of cilostamide on oocytes in this study was overcome by addition of FSH in culture (FIG. 8B). In the absence of FSH, 40-50% of cilostamide-treated oocytes remain arrested at the M I stage after 24 hours of culture (FIG. 8A). However, the addition of FSH, whilst initially delaying GVBD (FIG. 5), eventually induced the majority of cilostamide-treated oocytes to progress to M II after 24 hours of culture (FIG. 8B). The exception were oocytes collected in media supplemented with cAMP modulators during the pre-IVM phase, where even in the presence of FSH there was a delay in GVBD. However, oocytes treated in this way eventually progressed to the M II stage after 28 hours of culture (FIG. 8B), indicating that the efficacy of including cAMP modulators in IVM media can be enhanced by modulating COC cAMP levels following collection, thereby leading to further meiotic inhibition which may be induced by the presence of FSH in culture.

This observation may be the first model described in a ruminant species for induced oocyte maturation, as opposed to spontaneous maturation where mechanically removing oocytes from their follicle results in spontaneous meiotic resumption. However, although it is well established that in vivo, meiotic resumption occurs due to the pre-ovulatory surge of gonadotrophins, the mechanism(s) by which this occurs is not fully understood.

When oocytes were cultured in the presence of cilostamide, FSH treatment resulted initially in an inhibitory effect that later becomes stimulatory (FIG. 8). It has been proposed that cAMP generated by FSH has a paradoxical effect in that it initially blocks maturation before downstream participants in the signal cascade eventually exert a positive influence. To test if EGF-like peptides could be responsible for this observation, the experiment of induced IVM was repeated, with FSH treatment, and also with exposure to increasing doses of the EGFR tyrosine kinase inhibitor, AG1478. Increasing doses of AG1478 completely prevented the meiotic recovery of FSH and cilostamide treated COCs, indicating that the synthesis and release of EGF-like peptides and EGF signalling is required for FSH-induced oocyte maturation.

The experiments reported here also show that modulating oocyte cAMP levels during pre-IVM and IVM increased the capacity to support early development. As shown in Table 2, modulating cAMP during pre-IVM alone, or during IVM alone, generated a blastocyst percentage around 30%. However, including cAMP modulators in both phases led to a significant increase (up to 3 fold) in blastocyst percentage (69%). It is interesting to note that in the absence of cAMP modulators in the pre-IVM, and culturing the oocytes in the presence of cilostamide decreased blastocyst percentage (29%) compared to their presence in the pre-IVM media (69%). These current results are in parallel with previous reports on coasting in vivo, in which 80% blastocysts have been developed, when animals received leutinising hormone 6 hours before oocyte aspiration.

Collectively, the evidence presented in this example demonstrated, for the first time, that intracellular cAMP levels during the pre-IVM phase has a profound effect on oocyte developmental competence and blastocyst quality, starting from COC removal from the follicle to the end of maturation. A stable modulation of intracellular cAMP levels within a physiological range in the oocyte and cumulus cells during in vitro maturation can create an induced maturation in vitro that in turn can lead to acquisition of high developmental potential.

### Example 2

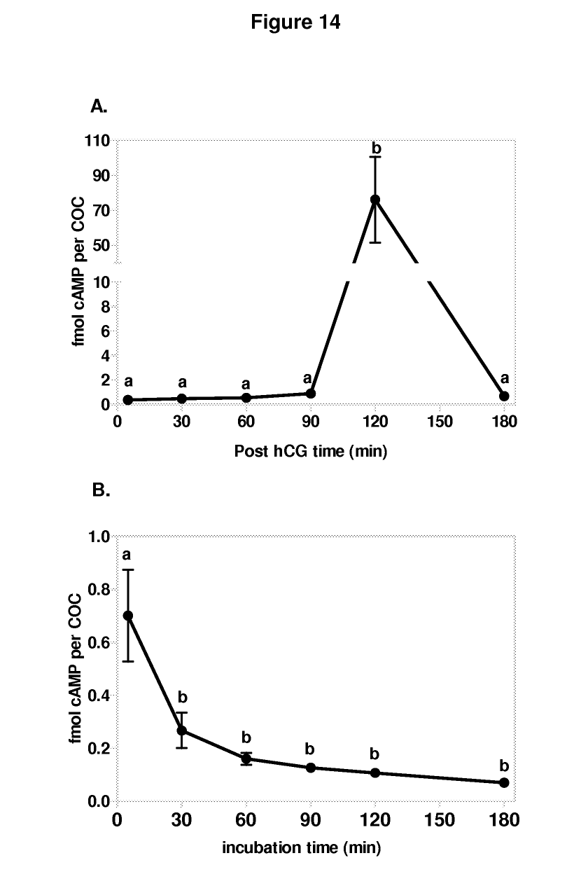

FIG. 14 shows the results of a comparison between in vivo cAMP concentrations in mouse cumulus-oocyte complexes (COCs) following follicular growth induced by equine chorionic gonadotropin (eCG) and human chorionic gonadotropin (hCG)-induced oocyte maturation (A) compared with cAMP concentrations in in vitro cultured COCs during pre-IVM phase with no cAMP modulators (2 hours which includes COO collection and selection) before oocyte in vitro maturation with no cAMP modulators (B). The data represents the mean cAMP level per COC±SEM of 4 replicates. Each measurement was conducted on 12 COCs.

The figure shows that there was a significant increase in cAMP concentration in in vivo matured oocytes following the hCG treatment, whereas spontaneously matured in vitro matured oocytes had low levels of cAMP, which fall even further. Such a loss in cAMP with spontaneously matured oocytes appeared to be associated with reduced developmental competence.

### Example 3

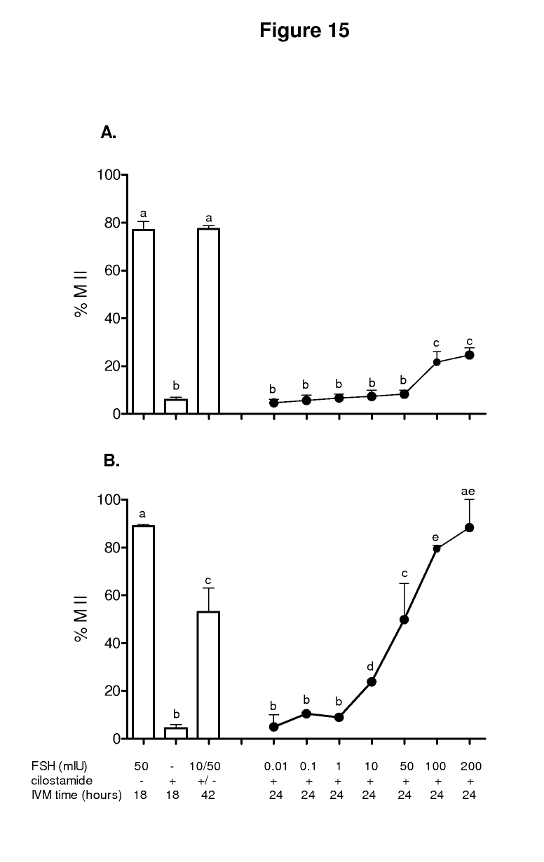

FIG. 15 shows the effect of increasing doses of FSH to induce meiotic maturation of mouse cumulus-oocyte complexes (COCs) matured in the presence of the type-3 PDE inhibitor (cilostamide; 1 μM (A) or 0.1 μM (B)). In this example, the basic medium used for mouse oocyte collection (without cyclic AMP modulators) was a HEPES-buffered α-Minimal Essential Medium (HEPES-MEM), supplemented with 3 mg/ml BSA, 1 mg/ml fetuin and 1.0 or 0.1 μM cilostamide, depending on what the concentration of cilostamide was used for IVM. COCs were held in the collection medium for approximately 30 minutes. For IVM, the basic maturation medium was a bicarbonate buffered α-Minimal Essential Medium (MEM) supplemented with 3 mg/ml BSA, 1 mg/ml fetuin and incubated at 37° C. in humidified 6% CO₂ in air. Variable FSH concentrations were used. COCs were cultured in MEM+cilostamide (1 μM (A) or 0.1 μM (B)) and increasing doses of FSH (0.01-200 mlU) for 24 hours of IVM. Oocytes were then fixed and assessed for meiotic progression at 18, 24 and 42 hours. A mean number of 40 oocytes were used in each treatment group and time-point from four replicate experiments. The bars in FIGS. 15A and 15B represent further control groups as follows. Bar 1: COCs matured with 50 mlU FSH alone (18 hours of IVM); Bar 2: COCs matured with 1 μM (A) or 0.1 μM (B) cilostamide alone (18 hours of IVM); and Bar 3: COCs matured with (1 μM (A) or 0.1 μM (B) cilostamide and 10 mlU FSH for 24 hours and then matured with 50 mlU FSH for 18 hours of IVM (bi-phasic IVM).

This figure demonstrates that the concentration of both PDE inhibitor and FSH together can regulate the timing of induced maturation.

### Example 4

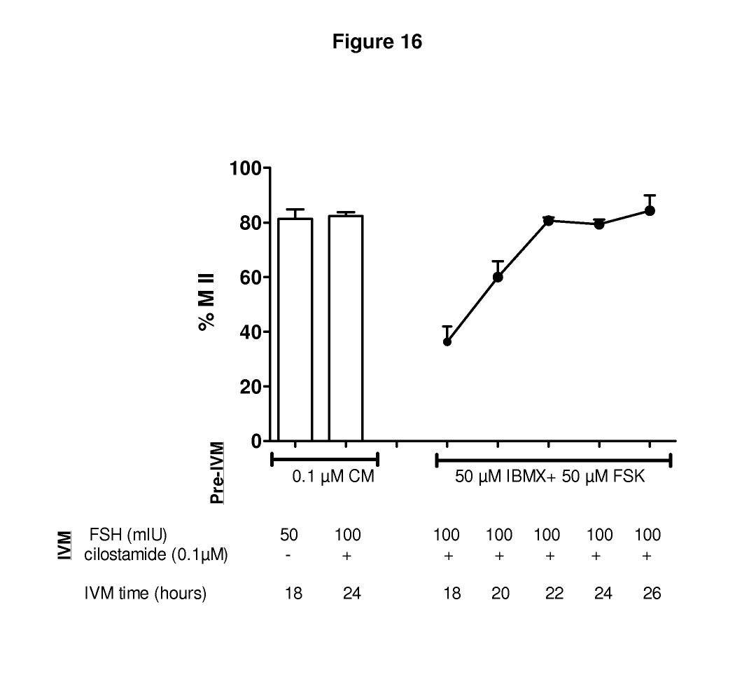

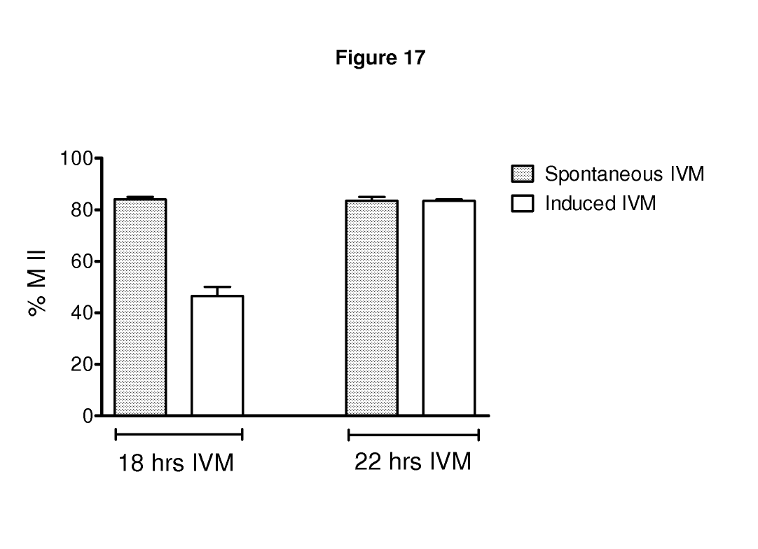

FIG. 16 shows the effect of cAMP modulators during both pre-IVM and IVM phases on oocyte meiotic resumption. In this example, the basic medium used for mouse oocyte collection (without cyclic AMP modulators) was a HEPES-buffered α-Minimal Essential Medium (HEPES-MEM), supplemented with 3 mg/ml BSA, 1 mg/ml fetuin. For IVM, the basic maturation medium was bicarbonate buffered α-Minimal Essential Medium (MEM) supplemented with 3 mg/ml BSA, 1 mg/ml fetuin and incubated at 37° C. in humidified 6% CO₂ in air. COCs were aspirated and selected in either collection medium supplemented with 0.1 μM cilostamide or collection medium supplemented with 50 μM forskolin (FSK); and 50 μM IBMX for 1 hour. COCs were then matured in maturation medium in the presence of 100 mlU FSH and 0.1 μM cilostamide for 18-26 hours. Oocytes were then fixed and assessed for meiotic progression at each time point. A mean number of 45 oocytes were used in each treatment group and time-point from four replicate experiments. Therefore optimal time for insemination to fertilize mouse eggs under the induced maturation conditions described here is 22 hours and therefore delayed from the “normal practice” of 18 hours (as demonstrated by the 1^{st} bar see of FIG. 17).

### Example 5

FIG. 17 is a graph showing the meiotic maturation of mouse COCs matured either in induced IVM (collection=HEPES-MEM+forskolin+IBMX, maturation=MEM+cilostamide+FSH) or spontaneous IVM (collection=HEPES-MEM, maturation

=MEM+FSH) following 18 and 22 hours of culture. Oocytes were then assessed for meiotic progression and classified as MII stage.

FIG. 17 shows that meiotic maturation of mouse COCs is delayed when matured in induced IVM at 18 hours when compared with spontaneous IVM, but that the rates of MII are the same and equivalent to spontaneous IVM at 22 hours maturation. This demonstrates that induced maturation slows down the progression of maturation in the mouse.

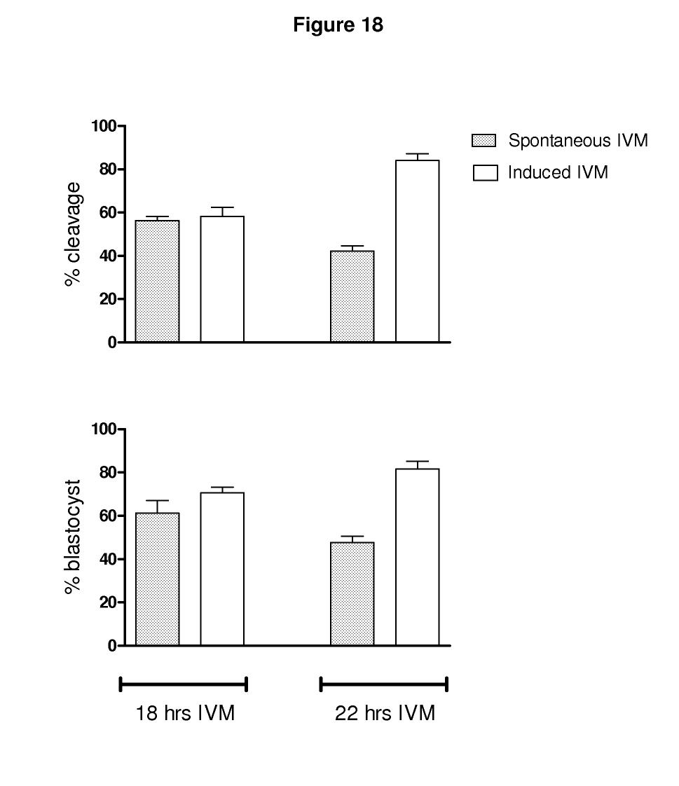

As shown in FIG. 18, this subsequently improves the developmental competence (capacity) of oocytes, demonstrated by the increased development of fertilized zygotes. Oocyte developmental capacity was assessed after in vitro fertilization and embryo development by cleavage rate (day 2)(A) and blastocyst rate (day 5)(B). Induced IVM (collection=HEPES-MEM+forskolin+IBMX, maturation=MEM+cilostamide+FSH). Spontaneous IVM (collection=HEPES-MEM, maturation=MEM+FSH).

As seen in FIG. 18, rates of cleavage (top graph) and blastocyst yields (bottom graph) were highest in induced maturation, when matured for 22 hours compared with both systems following maturation for 18 hours or spontaneous maturation for 22 hours.

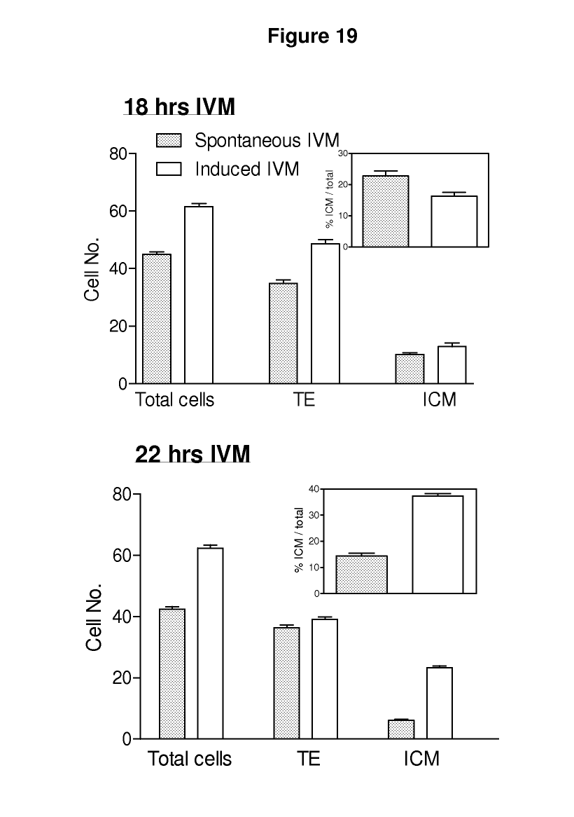

This improved oocyte developmental competence is also reflected in blastocyst quality as shown in FIG. 19. COCs were matured using either induced or spontaneous IVM for 18 (top graph) or 22 (bottom graph) hours. Matured oocytes were then fertilized. Embryos were cultured for 5 days and blastocyst quality was quantified by total cell counts (“Total cells”) and cell allocation to trophoectoderm (TE) or inner cell mass (ICM). Induced IVM (collection=HEPES-MEM+forskolin+IBMX, maturation=MEM+cilostamide+FSH). Spontaneous IVM (collection=HEPES-MEM, maturation=MEM+FSH).

As can be seen from FIG. 19, the number of total cells, inner mass cells (ICM), and the proportion of inner mass cells to total cells, after maturation for 22 hours in the induced maturation system, was greater than that for the spontaneous maturation system. The results demonstrate that, for the mouse oocyte, these aforementioned benefits occur when the collection medium comprises the PDE inhibitor IBMX.

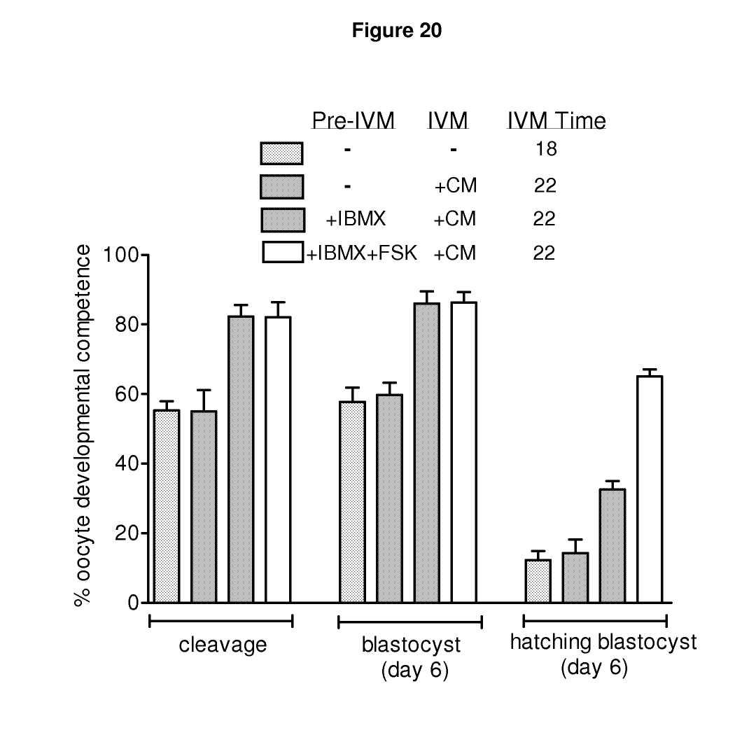

However, as shown in FIG. 20, these benefits are further improved when both forskolin and IBMX are included in the collection medium. COCs were subjected to incubation for 1 hour in either basic collection medium or collection medium supplemented with 50 μM IBMX+/−50 μM forskolin (FSK). COCs were then matured in MEM+50 mlU FSH either without cilostamide for 18 hours or for 22 hours in MEM+100 mlU FSH in the presence of cilostamide (0.1 μM). Subsequently, oocyte developmental capacity was assessed after in vitro fertilization and embryo development by cleavage rate, blastocyst rate and hatching blastocyst rate on day 6.

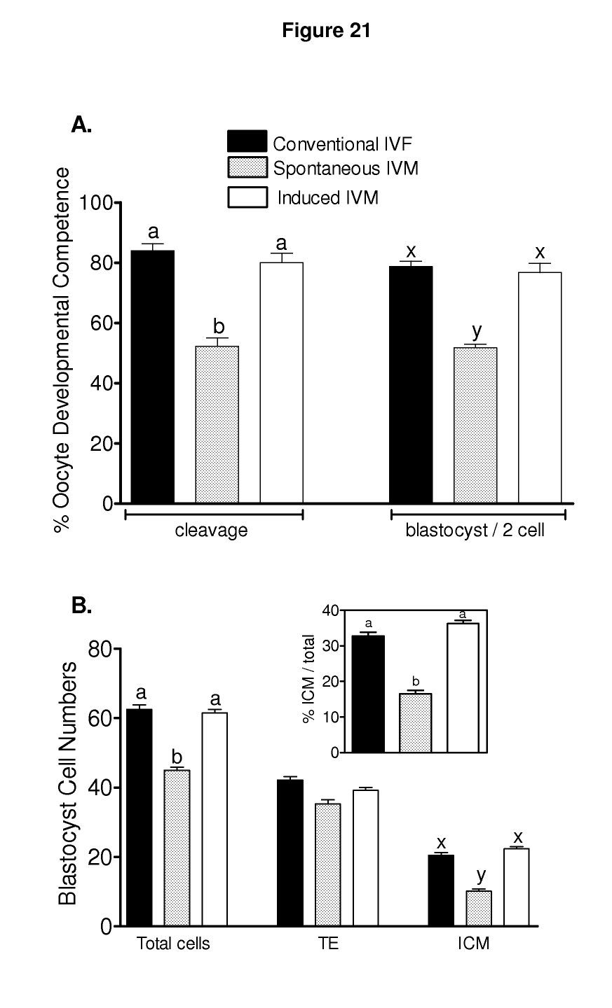

FIG. 21 are graphs showing the developmental competence of oocytes matured in vivo, either by induced IVM (collection=HEPES-MEM+forskolin+IBMX, maturation=MEM+cilostamide+FSH) or by spontaneous IVM (collection=HEPES-MEM, maturation=MEM+FSH). Following IVM, COCs were fertilized and embryos were cultured until day 5 (A). Blastocyst quality was quantified by total cell counts and cell allocation to trophoectoderm (TE) or inner cell mass (ICM) (B). In vivo matured control oocytes=COCs collected from oviducts 14 hours after hCG administration.

FIG. 21 demonstrates that only induced IVM, not spontaneous IVM, produces results (in terms of oocyte developmental competence) that closely mimic oocytes obtained from ovulated follicles (and have acquired full developmental competence, referred to here as in vivo matured oocytes).

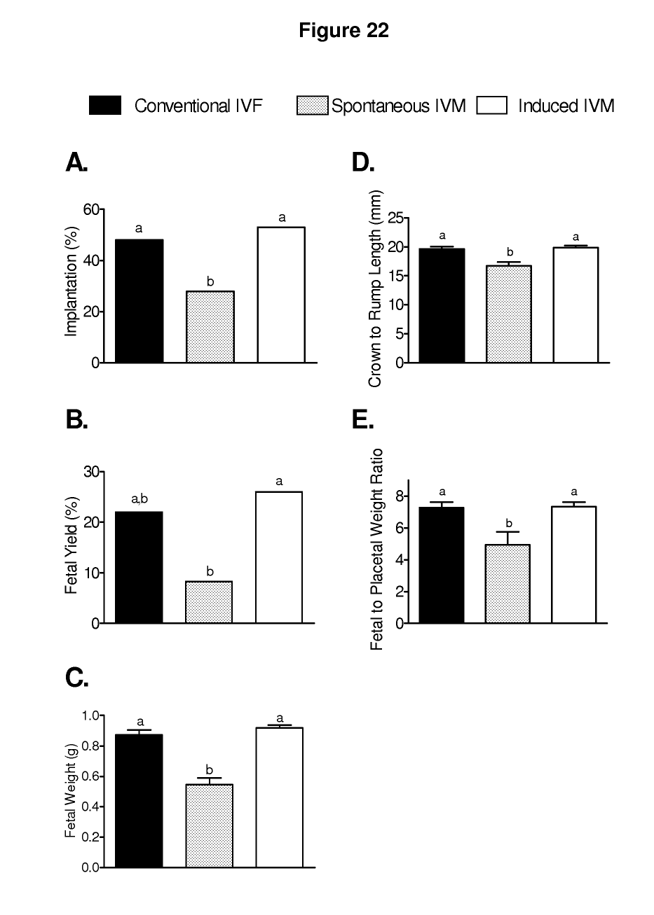

FIG. 22 provides graphs showing the effect of induced IVM on pregnancy outcomes and fetal parameters. Day 4.5 blastocysts developed from COCs matured in vivo, by induced IVM (collection=HEPES-MEM+forskolin+IBMX, maturation=MEM+cilostamide+FSH) or by spontaneous IVM (collection=HEPES-MEM, maturation=MEM+FSH) were transferred to pseudo-pregnant recipients and outcomes analysed on day 18 of pregnancy. Implantation rate=total implantations/embryos transferred. Fetal survival=number of fetuses/implantation site. In vivo matured control=COCs collected from oviducts 14 hours after hCG administration.

As can be seen in FIG. 22A-C, when embryos from the three different groups of oocyte maturation systems are transferred, embryos from induced IVM mimic the post-transfer developmental results obtained from in vivo matured oocytes, and are higher than that for embryos from spontaneously matured oocytes. This observation is also reflected in the size of the fetus (the crown-rump length) and in the fetal:placental weight ratio, as shown in FIG. 22D-E.

### Example 6

The concept of Induced-IVM developed using animal oocytes was then examined using human oocytes.

Adaptation of the technology to human requires determination of: (1) optimal concentration of cilostamide to use during a human IVM phase; (2) optimal concentrations of forskolin and IBMX to use during a human pre-IVM phase; (3) the interacting effects of these agents on the duration required to complete oocyte maturation; and (4) the effect of denuding time (somatic cell removal) on post-fertilisation embryo development.

### Materials and Methods

Immature human cumulus-oocyte complexes (COC) were collected from young, healthy women (not from IVF patients). These women received minimal ovarian stimulation (typically 300-500 lU FSH/cycle, no hCG), as is typically conducted in one variant of a routine clinical IVM cycle. Oocyte pick-up was performed when the lead ovarian follicle reached 12 mm. Immature oocytes were then stratified to various treatments during either the Pre-IVM or IVM periods, as detailed below. In addition, time of removal of somatic cells from oocytes (denuding) during the oocyte maturation period was examined. The following trials were conducted:
Dose of PDE3-inhibitor (cilostamide) during IVM: 0 versus 0.1 versus 1.0 μM
cAMP modulators during Pre-IVM: control versus IBMX versus IBMX+forskolin
Effect of time of oocyte denuding on embryo development: 30 versus 40 versus 44-48 hours

At the oocyte pick-up, COC were collected immediately into pre-IVM treatments in VitroCollect medium (Cook Australia, Brisbane, Australia) and held for one hour. Following pre-IVM, COC were washed and then transferred to IVM treatments (e.g. dose of cilostamide) in VitroMat medium (Cook) and matured under standard conditions. At the various times post-denuding, oocytes were monitored for maturation (polar body [PB] extrusion). At the end of maturation (48 hours) a final meiosis score was given. As oocytes matured they were inseminated using standard intra-cytoplasmic sperm injection (ICSI) procedures using donor sperm. 24 hours later fertilisation rates were determined. Embryos were cultured for up to 6 days until developmental arrest using standard procedures.

### Results

### Dose of Cilostamide During Human IVM

The results are shown below in Table 3.

TABLE 3

| | CilostamideTreatment | | |
|---|---|---|---|
| | 0 μm | 0.1 μM | 1.0 μM |
| | (n = 41) | (n = 39) | (n = 38) |
| Germinal Vesicle Breakdown | 54% | 44% | 13% |

Germinal Vesicle Breakdown (GVBD) is a marker that oocytes have resumed meiosis. These results show that 1.0 μM is an inhibiting dose of cilostamide for human oocytes. 0.1 μM is non-inhibiting so this dose was used during IVM for the study reported immediately below.

Effect of Pre-IVM cAMP Modulating Agents with 0.1 μM Cilostamide During Human IVM

The results are shown below in Tables 4 to 6.

TABLE 4

Pre-IVM: Control

| | n | % GV | % GVBD | % PB |
|---|---|---|---|---|
| 30 hours | 33 | 33.3 | 51.5 | 15.2 |
| 36-40 hours | 36 | 30.6 | 36.1 | 33.3 |
| 42-44 hours | 36 | 30.6 | 33.3 | 36.1 |
| 48 hours | 36 | 27.8 | 30.6 | 41.7 |

TABLE 5

Pre-IVM: IBMX

| | n | % GV | % GVBD | % PB |
|---|---|---|---|---|
| 30 hours | 27 | 11.1 | 48.1 | 40.7 |
| 36-40 hours | 39 | 10.3 | 43.6 | 46.2 |
| 42-44 hours | 32 | 9.4 | 28.1 | 62.5 |
| 48 hours | 39 | 10.3 | 28.2 | 61.5 |

TABLE 6

Pre-IVM: IBMX + FSK

| | n | % GV | % GVBD | % PB |
|---|---|---|---|---|
| 30 hours | 34 | 35.3 | 41.2 | 23.5 |
| 36-40 hours | 37 | 29.7 | 24.3 | 43.2 |
| 42-44 hours | 37 | 24.3 | 16.2 | 56.8 |
| 48 hours | 37 | 24.3 | 8.1 | 64.9 |

Note:
GV—germinal vesicle (immature);
GVBD—germinal vesicle breakdown (maturing);
PB—polar body (mature)

These results demonstrate the principal of Induced-IVM using human oocytes. The results show that, when oocytes are treated during the IVM phase with 0.1 μM cilostamide in the presence of FSH, Pre-IVM with IBMX+FSK increases the proportion of mature (64.9% PB-stage) oocytes relative to standard (the control; 41.7% PB-stage) pre-IVM oocyte collection conditions.

### Effect of Time of Denuding on Post-IVM Human Embryo Development

The results are shown below in Table 7.

TABLE 7

| Denuding | | | | | % Blast/Cleave | |
|---|---|---|---|---|---|---|
| Time | n PB | Fert % (n) | % Blast/PB (n) | | (n) | |
| 30 | 29 | 90% (26) | 35% | (10) | 39% | (10) |
| 40 | 25 | 96% (24) | 36% | (9) | 38% | (9) |
| 44 + 48 | 18\hat{ } | 72% (13) | 6% | (1) | 8% | (1) |

Note:
PB = polar body; Fert = Fertilisation; Blast = blastocyst; Cleave = cleaved embryo

Here we have generated human embryos from oocytes matured using the Induced-IVM system. Despite low numbers so far, the system is proving highly efficient at generating embryos with between 35-40% progressing to the blastocyst stage. This is substantially higher than the standard clinical rates. A critical factor in the Induced-IVM system is the duration of IVM or the time of insemination. The results here suggest that denuding between 44-48 hours adversely affects embryo development.

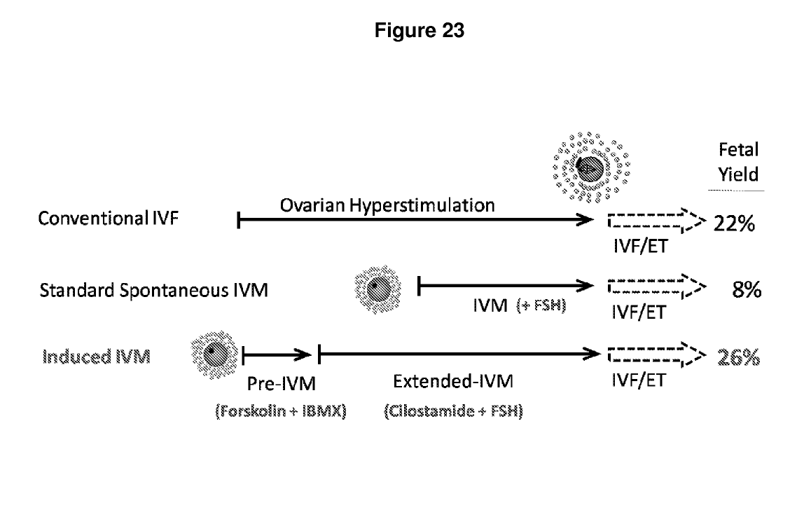

FIG. 23 is an illustration summarising the key concepts of induced IVM compared to conventional IVF (in vivo matured oocytes) and standard spontaneous IVM and the relative efficiencies of these three procedures at generating fetuses.

Finally, it will be appreciated that various modifications and variations of the methods and compositions of the invention described herein will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are apparent to those skilled in the art are intended to be within the scope of the present invention.

1. A method of producing an embryo from an oocyte by an assisted reproduction technology, the method comprising:
(a) collecting an oocyte from an ovary of a subject in a collection medium comprising a first phosphodiesterase inhibitor and an agent that increases intracellular cAMP concentration in the oocyte;
(b) culturing the oocyte in a maturation medium comprising a second phosphodiesterase inhibitor; and
(c) producing an embryo from the oocyte by an assisted reproduction technology.

2. The method according to claim 1, wherein the agent increases intracellular cAMP production in the oocyte or decreases intracellular cAMP degradation in the oocyte.

3. The method according to claim 2, wherein the agent is forskolin.

4. (canceled)

5. The method according to claim 1, wherein the method further includes exposing the oocyte to a ligand to induce maturation of the oocyte.

6. The method according to claim 5, wherein the concentration of the ligand overcomes cAMP-induced meiotic arrest of the oocyte.

7. The method according to claim 5, wherein the ligand is Follicle Stimulating Hormone (FSH) or Epidermal Growth Factor (EGF).

8-11. (canceled)

12. The method according to claim 1, wherein the first phosphodiesterase inhibitor is IBMX.

13. The method according to claim 1, wherein the second phosphodiesterase inhibitor is cilostamide.

14-18. (canceled)

19. A method of in vitro maturation of an oocyte, the method comprising:
(a) collecting an oocyte from an ovary of a subject in a collection medium comprising a first phosphodiesterase inhibitor and an agent that increases intracellular cAMP concentration in the oocyte; and
(b) culturing the oocyte in a maturation medium comprising a second phosphodiesterase inhibitor.

20. The method according to claim 19, wherein the agent increases intracellular cAMP production in the oocyte or decreases intracellular cAMP degradation in the oocyte.

21. The method according to claim 20, wherein the agent is forskolin.

22. (canceled)

23. The method according to claim 19, wherein the method further includes exposing the oocyte to a ligand to induce maturation of the oocyte.

24. The method according to claim 23, wherein the concentration of the ligand overcomes cAMP-induced meiotic arrest of the oocyte.

25. The method according to claim 24, wherein the ligand is Follicle Stimulating Hormone (FSH) or Epidermal Growth Factor (EGF).

26-29. (canceled)

30. The method according to claim 19, wherein the first phosphodiesterase inhibitor is IBMX.

31. The method according to claim 19, wherein the second phosphodiesterase inhibitor is cilostamide.

32. (canceled)

33. An oocyte maturation medium, the medium comprising
(a) a phosphodiesterase inhibitor; and
(b) a ligand for inducing maturation of the oocyte,
wherein the concentration of the ligand in the oocyte maturation medium overcomes cAMP-induced meiotic arrest of the oocyte.

34. The oocyte maturation medium according to claim 33, wherein the phosphodiesterase inhibitor is cilostamide.

35. The oocyte maturation medium according to claim 33, wherein the ligand is FSH or EGF.

36-40. (canceled)

41. A combination product comprising the following components:
(a) an oocyte collection medium comprising a first phosphodiesterase inhibitor and an agent that increases intracellular cAMP concentration in the oocyte; and
(b) an oocyte maturation medium comprising a second phosphodiesterase inhibitor and a ligand for inducing maturation of the oocyte;
wherein the concentration of the ligand in the oocyte maturation medium overcomes cAMP-induced meiotic arrest of the oocyte.

42. The combination product according to claim 41, wherein the collection medium and the maturation medium are used for collection and maturation of a human oocyte or a bovine oocyte.

43. (canceled)

44. A method of inducing oocyte maturation, the method comprising culturing an oocyte in a maturation medium comprising a phosphodiesterase inhibitor and a ligand for inducing maturation of the oocyte, wherein the concentration of the ligand in the maturation medium overcomes cAMP-induced meiotic arrest of the oocyte, thereby maturing the oocyte.

45. A method of inducing maturation of an oocyte which is in a state of meiotic arrest, the method comprising contacting the oocyte with a ligand at a concentration sufficient to overcome the meiotic arrest.

46-47. (canceled)
